SEQUENCE LISTING (43 sequences)

SEQ ID NO: 1 - DNA, 1368 bp, Homo sapiens
  CDS (1)..(1365)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 2 - protein, 455 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1476 bp, Homo sapiens
  CDS (1)..(1476)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctgaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 492 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEQKLISEEDLKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 1590 bp, Homo sapiens
  CDS (1)..(1590)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - protein, 530 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITRSKRSRLLHSDYMNMTPRRPGPTRKHYQPYAPPRDFAAYRSRDQRLPPDAHKPPGGGSFRTPIQEEQADAHSTLAKIRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 1608 bp, Homo sapiens
  CDS (1)..(1608)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccacccgatgcccacaagccccctggaggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 536 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 1368 bp, Homo sapiens
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtactagaggctattctccttattcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctggaacgttcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 10 - protein, 455 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSPYSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSWNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1510 bp, Homo sapiens
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtactagaggctattctccttattcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctggaacgttcctcttacattcggtgatggcaccaagcttgaaaaagctctagagcagaaactgatctcggaagaagatctgaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagcggccctagcgcggccctagctcgacatga

SEQ ID NO: 12 - protein, 492 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSPYSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSWNVPLTFGDGTKLEKALEQKLISEEDLKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 1590 bp, Homo sapiens
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtactagaggctattctccttattcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctggaacgttcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 530 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSPYSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSWNVPLTFGDGTKLEKALEQKLISEEDLAKPTTTPAPRPPTPAPTIASQPLSLRPEACRPAAGGAVHTRGLDFACDIYIWAPLAGTCGVLLLSLVITRSKRSRLLHSDYMNMTPRRPGPTRKHYQPYAPPRDFAAYRSRDQRLPPDAHKPPGGGSFRTPIQEEQADAHSTLAKIRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1608 bp, Homo sapiens
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtactagaggctattctccttattcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctggaacgttcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccacccgatgcccacaagccccctggaggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 536 aa, Homo sapiens
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSPYSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSWNVPLTFGDGTKLEKALEQKLISEEDLAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 9 aa, Artificial Sequence
  Description of Artificial Sequence Synthetic peptide
EIKLISEED

SEQ ID NO: 18 - DNA, 22 bp, Artificial Sequence
  Description of Artificial Sequence Synthetic primer
cgtgcctcttacattcggtgat

SEQ ID NO: 19 - DNA, 22 bp, Artificial Sequence
  Description of Artificial Sequence Synthetic primer
cctccgccatcttatctttctg

SEQ ID NO: 20 - DNA, 14 bp, Artificial Sequence
  Description of Artificial Sequence Synthetic primer
ctctcctggtaacc

SEQ ID NO: 21 - DNA, 22 bp, Artificial Sequence
  Description of Artificial Sequence Synthetic primer
cctccgccatcttatctttctg

SEQ ID NO: 22 - DNA, 22 bp, Artificial Sequence
  Description of Artificial Sequence Synthetic primer
cgtgcctcttacattcggtgat

SEQ ID NO: 23 - DNA, 30 bp, Artificial Sequence
  Description of Artificial Sequence Synthetic primer
tatcgctcagcgcgcgggaggctctgcgct

SEQ ID NO: 24 - DNA, 22 bp, Artificial Sequence
  Description of Artificial Sequence Synthetic primer
cctccgccatcttatctttctg

SEQ ID NO: 25 - DNA, 29 bp, Artificial Sequence
  Description of Artificial Sequence Synthetic primer
tgacggggtcacccacactgtgcccatct

SEQ ID NO: 26 - DNA, 26 bp, Artificial Sequence
  Description of Artificial Sequence Synthetic primer
cgaagcattgcggtggacgatggagc

SEQ ID NO: 27 - protein, 9 aa, Artificial Sequence
  Description of Artificial Sequence Synthetic peptide
EDKLISEED

SEQ ID NO: 28 - protein, 22 aa, Homo sapiens
MDWIWRILFLVGAATGAHSQVQ

SEQ ID NO: 29 - protein, 21 aa, Homo sapiens
MALPVTALLLPLALLLHAARP

SEQ ID NO: 30 - protein, 18 aa, Homo sapiens
MLRLLLALNLFPSIQVTG

SEQ ID NO: 31 - protein, 21 aa, Homo sapiens
MKWKALFTAAILQAQLPITEA

SEQ ID NO: 32 - DNA, 1400 bp, Artificial Sequence
  Description of Artificial Sequence Synthetic polynucleotide
tctagaatggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagcggccctagcgcggccctagctcga

SEQ ID NO: 33 - DNA, 1400 bp, Artificial Sequence
  Description of Artificial Sequence Synthetic polynucleotide
tcgagctagggccgcgctagggccgctcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcggggggctctgcgctcctgctgaacttcactctgtaaagggtgataaccagtgacaggagaaggaccccacaagtcccggccaggggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggtgggcttcgccagatcttcttccgagatcagtttctgctctagagctttttcaagcttggtgccatcaccgaatgtaagaggcacgttaaagctttgcaaacagtagtaatctccaacatcttccgagagtgtgttttcaattgtaaaaacaaaatctgtgccagtgccactgctggaaaatcgggatgggactcccggccgaagagtattgccttctgaaataaggaacttagggggttcccctggcttctgctggtaccagttcatatcatcatcaatatcagtgctggtcatgcatctgatagtgactttttctcctgtagccacggacagggatgctggagactgagtgagctcgatgtccgatccgccaccgccagagccacctccgcctgaaccgcctccacttgaggagacggtgaccgtggtcccttggccccagtagtccatagcataagaggtactagaatagcctcttgtacagtaatacaaggccgtgtcctcagacttcagactactcatttgcaggtacagggtgttcttggcattctctctggagatggtgaatcggccctttacattgtctgaatagtacgtgttataaccgccagtactaatggatgcgacccattccagcctcttgtcactagtctggcgaacccaagacatgccaaattttctgaaagtgaatccagaggttacacaggagagtttcagagacgctccaggcttcactaagcctcccccagactgctggagttgtacctgagaatgcgcgccggtagcagcgccgacaaggaaaaggatgcgccaaatccagtccattctaga

SEQ ID NO: 34 - DNA, 1600 bp, Artificial Sequence
  Description of Artificial Sequence Synthetic polynucleotide
tctagaatggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgag

SEQ ID NO: 35 - DNA, 1600 bp, Artificial Sequence
  Description of Artificial Sequence Synthetic polynucleotide
ctcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcggggggctctgcgctcctgctgaacttcactctgatcttggccagggtggagtgggcgtcggcctgctcctcttggatgggggtccggaaactgcctcccccagggggcttgtgggcatcggggggcagcctctggtccctggagcgataggctgcgaagtcgcgtggtggggcatagggctggtaatgcttgcgggtgggcccggggcggcggggagtcatgttcatgtagtcactgtgcaggagcctgctcctcttactcctggtgataaccagtgacaggagaaggaccccacaagtcccggccaggggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggtgggcttcgccagatcttcttccgagatcagtttctgctctagagctttttcaagcttggtgccatcaccgaatgtaagaggcacgttaaagctttgcaaacagtagtaatctccaacatcttccgagagtgtgttttcaattgtaaaaacaaaatctgtgccagtgccactgctggaaaatcgggatgggactcccggccgaagagtattgccttctgaaataaggaacttagggggttcccctggcttctgctggtaccagttcatatcatcatcaatatcagtgctggtcatgcatctgatagtgactttttctcctgtagccacggacagggatgctggagactgagtgagctcgatgtccgatccgccaccgccagagccacctccgcctgaaccgcctccacttgaggagacggtgaccgtggtcccttggccccagtagtccatagcataagaggtactagaatagcctcttgtacagtaatacaaggccgtgtcctcagacttcagactactcatttgcaggtacagggtgttcttggcattctctctggagatggtgaatcggccctttacattgtctgaatagtacgtgttataaccgccagtactaatggatgcgacccattccagcctcttgtcactagtctggcgaacccaagacatgccaaattttctgaaagtgaatccagaggttacacaggagagtttcagagacgctccaggcttcactaagcctcccccagactgctggagttgtacctgagaatgcgcgccggtagcagcgccgacaaggaaaaggatgcgccaaatccagtccattctaga

SEQ ID NO: 36 - DNA, 1700 bp, Artificial Sequence
  Description of Artificial Sequence Synthetic polynucleotide
tctagaatggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccacccgatgcccacaagccccctggaggaggcagtttccggacccccatccaagaggagcaggccgacgcccactccaccctggccaagatcagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagcggccctagcgcggccctagctcgacatgataagatacattgatgagtttggacaaaccacaactagaatgcagtgaaaaaa

SEQ ID NO: 37 - DNA, 1700 bp, Artificial Sequence
  Description of Artificial Sequence Synthetic polynucleotide
ttttttcactgcattctagttgtggtttgtccaaactcatcaatgtatcttatcatgtcgagctagggccgcgctagggccgctcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcggggggctctgcgctcctgctgaacttcactctgatcttggccagggtggagtgggcgtcggcctgctcctcttggatgggggtccggaaactgcctcctccagggggcttgtgggcatcgggtggcagcctctggtccctggagcgataggctgcgaagtcgcgtggtggggcatagggctggtaatgcttgcgggtgggcccggggcggcggggagtcatgttcatgtagtcactgtgcaggagcctgctcctcttactcctcacccagaaaataataaaggccactgttactagcaagctatagcaagccaggactccaccaaccaccaccagcacccaaaaatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggtgggcttcgccagatcttcttccgagatcagtttctgctctagagctttttcaagcttggtgccatcaccgaatgtaagaggcacgttaaagctttgcaaacagtagtaatctccaacatcttccgagagtgtgttttcaattgtaaaaacaaaatctgtgccagtgccactgctggaaaatcgggatgggactcccggccgaagagtattgccttctgaaataaggaacttagggggttcccctggcttctgctggtaccagttcatatcatcatcaatatcagtgctggtcatgcatctgatagtgactttttctcctgtagccacggacagggatgctggagactgagtgagctcgatgtccgatccgccaccgccagagccacctccgcctgaaccgcctccacttgaggagacggtgaccgtggtcccttggccccagtagtccatagcataagaggtactagaatagcctcttgtacagtaatacaaggccgtgtcctcagacttcagactactcatttgcaggtacagggtgttcttggcattctctctggagatggtgaatcggccctttacattgtctgaatagtacgtgttataaccgccagtactaatggatgcgacccattccagcctcttgtcactagtctggcgaacccaagacatgccaaattttctgaaagtgaatccagaggttacacaggagagtttcagagacgctccaggcttcactaagcctcccccagactgctggagttgtacctgagaatgcgcgccggtagcagcgccgacaaggaaaaggatgcgccaaatccagtccattctaga

SEQ ID NO: 38 - DNA, 1500 bp, Artificial Sequence
  Description of Artificial Sequence Synthetic polynucleotide
tctagaatggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctgaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagcggccctagcgcgg

SEQ ID NO: 39 - DNA, 1500 bp, Artificial Sequence
  Description of Artificial Sequence Synthetic polynucleotide
ccgcgctagggccgctcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcggggggctctgcgctcctgctgaacttcactctggagcgataggctgcgaagtcgcgtggtggggcatagggctggtaatgcttgcgggtgggcccggggcggcggggagtcatgttcatgtagtcactgtgcaggagcctgctcctcttactcctcacccagaaaataataaaggccactgttactagcaagctatagcaagccaggactccaccaaccaccaccagcacccaaaagggcttagaaggtccgggaaataggggacttggacaaaggtgtttccctttcacatggataatggttccattgctcttctcattgtctaggtaaggaggaggatacataacttcaattttcagatcttcttccgagatcagtttctgctctagagctttttcaagcttggtgccatcaccgaatgtaagaggcacgttaaagctttgcaaacagtagtaatctccaacatcttccgagagtgtgttttcaattgtaaaaacaaaatctgtgccagtgccactgctggaaaatcgggatgggactcccggccgaagagtattgccttctgaaataaggaacttagggggttcccctggcttctgctggtaccagttcatatcatcatcaatatcagtgctggtcatgcatctgatagtgactttttctcctgtagccacggacagggatgctggagactgagtgagctcgatgtccgatccgccaccgccagagccacctccgcctgaaccgcctccacttgaggagacggtgaccgtggtcccttggccccagtagtccatagcataagaggtactagaatagcctcttgtacagtaatacaaggccgtgtcctcagacttcagactactcatttgcaggtacagggtgttcttggcattctctctggagatggtgaatcggccctttacattgtctgaatagtacgtgttataaccgccagtactaatggatgcgacccattccagcctcttgtcactagtctggcgaacccaagacatgccaaattttctgaaagtgaatccagaggttacacaggagagtttcagagacgctccaggcttcactaagcctcccccagactgctggagttgtacctgagaatgcgcgccggtagcagcgccgacaaggaaaaggatgcgccaaatccagtccattctaga

SEQ ID NO: 40 - DNA, 6624 bp, Artificial Sequence
  Description of Artificial Sequence Synthetic oligonucleotide
tctagaatggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtactagaggctattctccttattcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctggaacgttcctcttacattcggtgatggcaccaagcttgaaaaagctctagagcagaaactgatctcggaagaagatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagcggccctagcgcggccctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttagcgaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaagctgcaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacctagttgtgaccggcgcctagtgttgacaattaatcatcggcatagtatatcggcatagtataatacgactcactataggagggccaccatgtcgactactaaccttcttctctttcctacagctgagatcaccggtaggagggccatcatgaaaaagcctgaactcaccgcgacgtctgtcgcgaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaacccgtcgcggagctcatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagtcgagaattcgctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcagggcccaattgctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacggattcatcgagagggacacgtcgactactaaccttcttctctttcctacagctgagatt

SEQ ID NO: 41 - protein, 12 aa, Artificial Sequence
  Description of Artificial Sequence Synthetic peptide
GGGSGGGSGGGS

SEQ ID NO: 42 - protein, 6 aa, Artificial Sequence
  Description of Artificial Sequence Synthetic 6x His tag
HHHHHH

SEQ ID NO: 43 - protein, 28 aa, Homo sapiens
MCVGARRLGRGPCAALLLLGLGLSTVTG

(19) United States
(12) Patent Application Publication
(10) Pub. No.: US 2010/0105136 A1
(43) Pub. Date: Apr. 29, 2010

(54) CHIMERIC T-CELL RECEPTORS AND T-CELLS TARGETING EGFRVIII ON TUMORS

(75) Inventors: Bob S. Carter, Belmont, MA (US); Richard C. Mulligan, Cambridge, MA (US); Szofia Bullain, Brookline, MA (US)

Correspondence Address: DAVID S. RESNICK, NIXON PEABODY LLP, 100 SUMMER STREET, BOSTON, MA, 02110-2131, US

(73) Assignees: The General Hospital Corporation, Boston, MA (US); Children's Medical Center Corporation, Boston, MA (US)

(21) Appl. No.: 12/444,090

(22) PCT Filed: Oct. 9, 2007
(86) PCT No.: PCT/US07/21575

§ 371 Date: Apr. 2, 2009

Related U.S. Application Data
(60) Provisional application No. 60/850,040, filed on Oct. 9, 2006.

(51) Int. Cl. C12N 5/0783; C07H 21/04; C07K 14/725

(52) U.S. Cl. 435/372.3; 536/23.5; 530/350

(57) ABSTRACT

Chimeric T-cell receptor proteins have been produced in cells by the construction of nucleic acids and vectors, and the transfection of the vectors into cells. The chimeric proteins comprise, as an extracellular binding portion, a single chain antibody portion that binds to EGFRvIII, a transmembrane portion derived from human CD8α or CD28, and an intracellular signaling portion derived from human CD3ζ. The invention includes nucleic acids, vectors and cells associated with the production of the chimeric membrane protein, as well as methods to treat rumors bearing EGFRvIII, a mutant epidermal growth factor receptor.

### CROSS REFERENCE TO RELATED APPLICATIONS

This application claims benefit of U.S. provisional application No. 60/850,040 filed Oct. 9, 2006, the contents of which are incorporated herein by reference in its entirety.

### FIELD OF THE INVENTION

This invention relates to compositions and methods for the treatment for cancers, particularly gliomas, breast, lung, ovarian, head and neck, and bladder cancers. More particularly, the invention relates to compositions and methods for the treatment for cancers involving aberrant epidermal growth factor receptor (EGFR) signaling.

### BACKGROUND OF THE INVENTION

Glioblastoma multiforme (GBM) is the most aggressive form of the primary brain tumors known collectively as gliomas. Glioblastoma patients have a survival of approximately 15 months when treated by a regimen of maximal surgical resection, involved field radiation, and temozolamide chemotherapy. This low survival has prompted intense ongoing efforts to consider alternative therapeutic strategies, including immunotherapy. Adoptive transfer of a variety of effector cell types (e.g. cytokine activated peripheral blood mononuclear cells, expanded tumor infiltrating lymphocytes) has been considered, as well as vaccination based strategies designed to mobilize the CD8+ effector arm of the immune system. A treatment option that specifically targets tumor cells but not normal cells, should offer extended survival times for these patients.

### SUMMARY OF THE INVENTION

The present invention provides enetically modified T-cells, which produce humanized chimeric proteins that cause the T-cells to specifically bind to and kill EGFRvIII expressing cells, but not normal or wild type EGFR expressing cells.

In one embodiment, the humanized chimeric proteins of the invention comprise three protein portions: (1) an extracellular domain comprising an EGFRvIII binding portion, serving to target the humanized chimeric protein expressing T-cell to an EGFRvIII expressing cell; (2) a middle portion comprising a transmembrane domain (TM) derived from a human protein for anchoring the humanized chimeric protein to a T-cell expressing the chimeric protein. The middle portion also serves in part to connect the extracellular domain to the intracellular domain. The middle portion can also serve to extend the extracellular EGFRvIII binding domain away from the T-cell plasma membrane so that the extracellular EGFRvIII binding domain can interact with a target EGFRvIII. The second portion can also assist in the intracellular signaling of the T-cell following the binding of the extracellular EGFRvIII-binding domain, thus relaying the message of EGFRvIII binding from the exterior of the T-cell to the interior of the T-cell; and (3) an intracellular domain comprising the human T-cell receptor zeta chain (TCRζ also known as CD3ζ).

In one embodiment, the invention relates to a nucleic acid encoding a chimeric protein, the chimeric protein comprising several polypeptide portions: (a) an MR1 single chain antibody, the antigen binding portion, MR1 scFv (b) a human CD8α hinge and TM, and (c) a human T-cell receptor zeta chain (TCR ζ) intracellular domain. The antigen binding portion binds specifically to the EGFRvIII. SEQ. ID. No. 1 provides a nucleic acid encoding such a chimeric protein, MR1-CD8-ζ. SEQ. ID. No. 2 provides an amino acid sequence for MR1-CD8-ζ. In particular embodiments, the invention includes a nucleic acid encoding a chimeric protein comprising SEQ. ID. No. 2, and a nucleic acid comprising SEQ. ID. No. 1.

In another embodiment the invention relates to a nucleic acid encoding a chimeric protein, the chimeric protein comprising the polypeptide portions: (a) an MR1 single chain antibody, the antigen binding portion, MR1scFv, (b) the human CD28 protein fragment comprising an extracellular domain, a transmembrane domain, and an intracellular domain, and (c) a human T-cell receptor zeta chain (TCR ζ) intracellular domain. SEQ. ID. No. 3 provides a nucleic acid encoding such a chimeric protein, MR1-CD28-ζ. SEQ. ID. No. 4 provides an amino acid sequence for MR1-CD28-ζ. In particular embodiments, the invention includes a nucleic acid encoding a chimeric protein comprising SEQ. ID. No. 4, and a nucleic acid comprising SEQ. ID. No. 3.

In a further embodiment, the invention relates to a nucleic acid encoding a chimeric protein, the chimeric protein comprising (a) an MR1 single chain antibody, the antigen binding portion, MR1scFv, (b) a human CD8α hinge and TM, (c) a human CD28 intracellular domain, (d) the human OX40 intracellular domain, and (e) a human T-cell receptor zeta chain (TCR ζ) intracellular domain. SEQ. ID. No. 5 provides a nucleic acid encoding such a chimeric protein, MR1-CD8TM-CD28-OX40-ζ. SEQ. ID. No. 6 provides an amino acid sequence for MR1-CD8TM-CD28-OX40-ζ. In a particular embodiment, the invention includes a nucleic acid encoding a chimeric protein comprising SEQ. ID. No. 6. In another particular embodiment, the nucleic acid comprises SEQ. ID. No. 5.

In yet another embodiment, the invention relates to a nucleic acid encoding a chimeric protein, the chimeric protein comprising (a) an MR1 single chain antibody, the antigen binding portion, MR1scFv, (b) a human CD8α hinge region, (c) a human CD28 TM and intracellular domain, (d) a OX40 intracellular domain, and (e) a human T-cell receptor zeta chain (TCR ζ) intracellular domain. SEQ. ID. No. 8 provides a nucleic acid encoding such a chimeric protein, MR1-CD8-CD28TM-OX40-ζ. SEQ. ID. No. 7 provides an amino acid sequence for MR1-CD8-CD28TM-OX40-ζ. In one embodiment, the invention includes a nucleic acid encoding a chimeric protein comprising SEQ. ID. No. 8. In another embodiment, the nucleic acid comprising SEQ. ID. No. 7.

The invention includes nucleic acids encoding chimeric proteins, like those proteins described above, but each nucleic acid encodes a chimeric protein with an MR1-1 single chain antibody, antigen binding portion, substituted for the MR1 single chain antibody antigen binding portion. The antigen binding portion of MR1-1 is derived from MR1 through two mutations: ST→PY and F→V (at amino acids 98 and 99 of the V_{H} of MR1scFv and F→V at amino acid 92 of the V_{L} of MR1scFv). MR1-1 has higher binding affinity for the antigen EGFRVIII than its parent MR1.

One aspect of the invention relates to a nucleic acid encoding a chimeric protein, the chimeric protein comprising (a) an MR1-1 single chain antibody, the antigen binding portion, MR1-1scFv, (b) a human CD8α hinge and TM, and (c) a human T-cell receptor zeta chain (TCR ζ) intracellular domain. SEQ. ID. No. 9 provides a nucleic acid encoding such a chimeric protein, MR1-1-CD8-ζ. SEQ. ID. No. 10 provides an amino acid sequence for the chimeric protein, MR1-1-CD8-ζ. In one aspect, the invention includes a nucleic acid encoding a protein comprising SEQ. ID. No. 10, which is the amino acid sequence as in SEQ. ID. No:9, but with the amino acid changes indicated for the MR1-1scFv as shown in FIG. 6. FIG. 11 also shows amino acid sequence SEQ. ID. No. 10. Also included is a nucleic acid comprising SEQ. ID. No. 9 (FIGS. 10A-10B). The coding region for SEQ. ID. No. 9 is nucleotides 1-1374.

Another aspect of the invention relates to a nucleic acid encoding a chimeric protein, the chimeric protein comprising (a) an MR1-1 single chain antibody, the antigen binding portion, MR1-1scFv, (b) the human CD28 protein fragment comprising an extracellular domain, a transmembrane domain, and an intracellular domain, and (c) a human T-cell receptor zeta chain (TCR ζ) intracellular domain. SEQ. ID. No. 11 provides a nucleic acid encoding such a chimeric protein, MR1-1-CD28-ζ. SEQ. ID. No. 12 provides an amino acid sequence for the chimeric protein, MR1-1-CD28-ζ. The invention, in another aspect, includes a nucleic acid encoding a protein comprising SEQ ID No. 12, which is the same as SEQ ID No. 4, except for the amino acid differences in the MR1-1 portion, compared to the MR1 portion, as shown in FIG. 6. Another particular aspect, included is a nucleic acid comprising nucleotide sequence SEQ. ID. No. 11. The nucleotide sequence SEQ ID No. 11 is identical to SEQ ID No. 5, except that SEQ ID No. 11, instead of the nucleotide sequence of the single chain antibody portion for MR1, has the nucleotide sequence of the single chain antibody portion for MR1-1 shown in the nucleotide sequence in FIG. 10A.

Yet another aspect of the invention relates to a nucleic acid encoding a chimeric protein, the chimeric protein comprising (a) an MR1-1 single chain antibody, the antigen binding portion, MR1-1scFv, (b) a human CD8α hinge and TM, (c) a human CD28 intracellular domain, (d) a human OX40 intracellular domain, and (e) a human T-cell receptor zeta chain (TCR ζ) intracellular domain. SEQ. ID. No. 13 provides for a nucleic acid encoding such a chimeric protein, MR1-CD8TM-CD28-OX40-ζ. SEQ. ID. No. 14 provides an amino acid sequence for the chimeric protein, MR1-CD8TM-CD28-OX40-ζ. A particular aspect of this invention includes a nucleic acid encoding a protein comprising SEQ. ID. No. 14, which is the same as SEQ. ID. No. 6, except for the amino acid differences in the MR1-1 portion, compared to the MR1 portion, as shown in FIG. 6. Another particular aspect is a nucleic acid comprising nucleotide sequence SEQ. ID. No. 13. The nucleotide sequence SEQ. ID. No. 13 is identical to SEQ. ID. No. 5, except that SEQ. ID. No. 13, instead of the nucleotide sequence of the single chain antibody portion for MR1, has the nucleotide sequence of the single chain antibody portion for MR1-1 shown in the nucleotide sequence in FIG. 10A.

In still another aspect, the invention relates to a nucleic acid encoding a chimeric protein, the chimeric protein comprising (a) an MR1-1 single chain antibody, the antigen binding portion, MR1-1scFv, (b) a human CD8α hinge region, (c) a human CD28 transmembrane domain and intracellular domain, (d) a human OX40 intracellular domain, and (e) a human T-cell receptor zeta chain (TCR ζ) intracellular signaling portion. SEQ. ID. No. 15 provides a nucleic acid encoding such a chimeric protein, MR1-CD8-CD28TM-OX40-ζ. SEQ. ID. No. 16 provides an amino acid sequence for the chimeric protein, MR1-CD8-CD28TM-OX40-ζ. In a particular aspect, the invention provides a nucleic acid encoding a protein comprising SEQ. ID. No:16, which is the same as SEQ. ID. No:8, except for the amino acid differences in the MR1-1 portion, compared to the MR1 portion, as shown in FIG. 6. Another particular aspect of the invention includes a nucleic acid comprising nucleotide sequence SEQ. ID. No. 15. The nucleotide sequence SEQ. ID. No. 15 is identical to SEQ. ID. No. 7, except that SEQ. ID. No. 15, instead of the nucleotide sequence of the single chain antibody portion for MR1, has the nucleotide sequence of the single chain antibody portion for MR1-1 shown in the nucleotide sequence in FIG. 10A.

The invention also includes all of the nucleic acids described herein consisting of the specified nucleotide sequences.

Any of the chimeric proteins set forth herein can further comprise a c-myc epitope, or can comprise some other epitope, such as 6X-histidine, V5, thioredoxin, glutathione-S-transferase, c-Myc, VSV-G, HSV, and FLAG for use in detecting the expression of the chimeric protein per se and the expression of the chimeric protein on the surface of a cell in culture, in a tissue sample, or in an animal or human. Strategic positioning of epitopes within the extracellular region of the chimeric protein is useful for the latter situation.

Further embodiments of the invention include a cell comprising one or more of the chimeric proteins described herein (also, a population of cells, each comprising one or more of the chimeric proteins described herein), for example, T-cells. The invention also encompasses a composition comprising a T-cell (also, a population of T-cells) that comprises one or more of the chimeric proteins. This composition can contain, for example, one or more other types of cells, one or more cytokines, and/or components of medium used to sustain the life of cells. The T-cells can, in some cases, be isolated T-cells maintained outside the body. In some cases, the T-cells can be CD8+ human T-cells.

Cells of the invention include, for example, cells that contain (e.g., in the cell membrane) one or more of the proteins comprising amino acid sequences SEQ. ID. Nos. 2, 4, 6, 8, 10, 12, 14 or 16. Cells of the invention also include cells that contain one or more of the proteins consisting of SEQ. ID. Nos. 2, 4, 6, 8, 10, 12, 14 or 16.

Cells comprising one or more vectors described herein are also part of the invention. The cells can be those transfected with a vector, for example, a viral vector. Vectors can comprise RNA or DNA, for instance. Vectors can be naked nucleic acid (e.g., plasmids, viral nucleic acids, or engineered nucleic acids produced by replication means of viral origin), or can be viruses containing coat proteins as well as nucleic acid. Vectors can be combined in a composition with other materials, for example those that facilitate introduction into cells.

In one embodiment, the invention provides a method of treating an EGFRvIII-expressing cancer in a human, comprising administering to a human diagnosed with an EGFRvIII-expressing cancer a population of modified human T-cells expressing the chimeric proteins described herein. In another embodiment, the invention provides a method of treating an EGFRvIII-expressing cancer in a human, comprising removing T-cells from a human diagnosed with an EGFRvIII-expressing cancer, transfecting said T-cells with a vector comprising a nucleic acid encoding the chimeric proteins described herein thereby producing a population of modified human T cells, and administering the population of modified T-cells to the same human. The EGFRvIII-expressing cancer can be selected from a group consisting of glioma, breast, lung, prostate, head and neck, bladder and ovarian cancer.

### BRIEF DESCRIPTION OF THE DRAWINGS

FIG. 1A is a schematic illustration of the structure of the chimeric T-cell receptor MR1-CD8-ζ (“MR1-CIR” in FIG. 1A), which contains the full length MR1scFv, CD8α hinge and transmembrane (TM) domain, and TCR intracellular domain.

FIG. 1B is a schematic illustration of the structure of the chimeric T-cell receptor MR1-CD8-Delζ (“MR1delZ-CIR” in FIG. 1B), which is truncated with no intracellular signaling ITAMs (ITAM=immunoreceptor tyrosine-based activation motif) of the TCR ζ.

FIG. 1C is a schematic illustration of the structure of the chimeric T-cell receptor MR1-B-CD8-ζ (“MR-B-CIR” in FIG. 1C). This mutant is deleted for two high affinity EGFRvIII binding sites to provide a control which does not bind EGFRvIII.

FIG. 1D is a schematic illustration of the structure of the chimeric T-cell receptor MR1-CD28-ζ (“MR1-28z-CIR” in FIG. 1D).

FIG. 1E is a schematic illustration of the structure of the chimeric T-cell receptor MR1-CD8TM-CD28-OX40-ζ (“MR1-CD8TM-CD28-OX40-ZETA CIR” in FIG. 1E).

FIG. 1F is a schematic illustration of the structure of the chimeric T-cell receptor MR1-CD8-CD28TM-OX40-ζ (“MR1-CD8-CD28TM-OX40-zeta-CIR” in FIG. 1F).

FIG. 1G is a schematic illustration of the structure of the MR1-1 binding domain of the single-chain antibody (scFv) described by Beers et al. (Clin. Cancer Res. 6(7):2835-2843, 2000), at amino acids 98 and 99 of the V_{H} of MR1scFv and F→V at amino acid 92 of the V_{L} of MR1scFv.

FIG. 2 is a diagram showing a procedure for expansion of a population of human CIR T-cells.

FIG. 3A is an image of a western blot showing detection of expression of chimeric receptors under reducing conditions (right lane) and non-reducing conditions (left lane).

FIG. 3B shows DNA products on a gel following RT-PCR. RNA was isolated and then amplified with or without reverse transcription from expanded T-cells (lanes 2-4) or 293T cells (lanes 5-7). Lane 1.1-kb plus DNA ladder of molecular weight markers; lane 2. MR1-CIR, RTase(+); lane 3. β-actin, RTase(+); lane 4. MR1-CIR, RTase(−); lane 5. MR1-CIR, RTase(+); lane 6. β-actin, RTase(+); lane 7. MR1-CIR, RTase(−); lane 8. positive control (MR1-CIR plasmid PCR); and lane 9. DNA ladder of molecular weight markers. Lane 2 shows detection of appropriately sized 500 by amplification product from transcripts of the MR1-CIR gene 12 weeks after nucleofection of primary human peripheral blood mononuclear cells.

FIG. 3C is a graph of results of flow cytometric detection of the embedded myc epitope in CD8+ cells. The polyclonal expanded MR1-CIR population was stained with anti-c-myc antibody (FL2) 12 weeks post nucleofection.

FIG. 4A is a graph showing the results of a europium release cyotoxicity analysis of human MR1-CIR T-cells targeting EGFRvIII. Human MR1-CIR T-cells effectively lyse U87 glioma cells that express EGFRvIII (either U87-EGFRvIII or Gli36vIII) as compared to non-EGFRvIII-expressing cells.

FIG. 4B is a graph showing the results of a europium release cyotoxicity analysis of human T-cells transfected with control vector encoding a non-EGFRvIII binding CIR. These T-cells do not lyse target cells, even at high effector to target (E:T) ratios.

FIG. 4C is a bar graph comparing cytotoxicity results at three different E:T ratios for human T-cells transfected with the MR1, MRB (also, “MR1-B-CD8-ζ”) or MR1delZ T-cell receptor proteins. (See FIGS. 1A, 1C and 1B, respectively.) Both binding and signaling domains are required for CIR activity. MRB T-cells and MR1delZ T-cells which have defective binding and signaling, respectively, show inhibited cytolysis compared to full length MR1-CIR T-cells.

FIG. 5 is a bar graph of results from cytokine bead array analysis after co-incubation of MR1-CIR T-cells with U87EGFRvIII or U87 glioma cells. Fifty thousand U87 or U87EGFRvIII target tumor cells were co-incubated with 500,000 human T-cells expressing MR1, MRB, or MR1delZ for 72 hours. Cytokine concentrations in the medium were assayed with the BD Pharmingen human cytokine bead array analysis kit. Bars on the graph for each CIR plus target cell combination are in the order as shown, top to bottom, IL-2 through INF-γ.

FIG. 6 is a representation of the amino acid sequence of human MR1-CD8-ζ (SEQ. ID. No:2) and the nucleic acid encoding MR1-CD8-ζ (SEQ. ID. No:1). The nucleic acid construct comprising SEQ. ID. No. 1 is shown here as a double stranded DNA, the sense strand is SEQ. ID. No. 32 and the anti-sense strand is SEQ. ID. No. 33.

FIG. 7 is a representation of the amino acid sequence of human MR1-CD8TM-CD28-OX40-ζ (SEQ. ID. No. 6) and the nucleic acid encoding MR1-CD8TM-CD28-OX40-ζ (SEQ. ID. No. 5). The nucleic acid construct comprising SEQ. ID. No. 5 is shown here as a double stranded DNA, the sense strand is SEQ. ID. No. 34 and the anti-sense strand is SEQ. ID. No. 35.

FIG. 8 is a representation of the amino acid sequence of human MR1-CD8-CD28TM-OX40-ζ (SEQ. ID. No. 8) and the nucleic acid encoding MR1-CD8-CD28TM-OX40-ζ (SEQ. ID. No. 7). The nucleic acid construct comprising SEQ. ID. No. 7 is shown here as a double stranded DNA, the sense strand is SEQ. ID. No. 36 and the anti-sense strand is SEQ. ID. No. 37.

FIG. 9 is a representation of the amino acid sequence of human MR1-CD28-ζ (SEQ ID No. 4) and the nucleic acid encoding MR1-CD28-ζ (SEQ. ID. No. 3). The nucleic acid construct comprising SEQ. ID. No. 3 is shown here as a double stranded DNA, the sense strand is SEQ. ID. No. 38 and the anti-sense strand is SEQ. ID. No. 39.

FIG. 10A-10B is a representation of the nucleic acid construct (SEQ. ID. No. 40) comprising the nucleic acid encoding human MR1-1-CD8-ζ. The nucleic acid coding region is nucleotides 7-1374 (SEQ. ID. No. 9). The amino acid sequence of MR1-1-CD8-ζ is SEQ. ID. No. 10.

FIG. 11 is a representation of the amino acid sequence of human MR1-1-CD8-ζ (SEQ. ID. No. 10).

FIG. 12 shows the bioluminescence imaging (BLI) data of four mice implanted intracranially with U87 tumor cells expressing firefly luciferase. The BLI data correlated directly with the size of tumor in the mice.

FIG. 13 is the BLI data of individual mice implanted intracranially with U87vIIILuc tumor cells and treated intravenously with humanized MR1-B-CD8-ζ T-cells (FIG. 13A) or MR1-CD8-ζ T-cells (FIG. 13B).

FIG. 14 is the average BLI data of mice implanted intracranially with a mixture of U87vIIILuc tumor cell and humanized MR1-B-CD8-ζ T-cells or a mixture of U87vIIILuc tumor cell and humanized MR1-CD8-ζ T-cells.

FIG. 15 shows the percentage survival (in days post implantation) of NOD-SCID mice implanted with intracranially with U87vIIILuc tumor cells and treated intravenously with humanized MR1-B-CD8-ζ T-cells or MR1-CD8-ζ T-cells.

FIG. 16 shows the amino acid sequence of human MR1-CD8-ζ (SEQ. ID. No. 2) and the nucleic acid encoding MR1-CD8-ζ (SEQ. ID. No. 1).

FIG. 17 shows the amino acid sequence of human MR1-CD28-ζ (SEQ. ID. No. 4) and the nucleic acid encoding MR1-CD28-ζ (SEQ. ID. No. 3).

FIG. 18 shows the amino acid sequence of human MR1-CD8TM-CD28-OX40-ζ (SEQ. ID. No. 6) and the nucleic acid encoding MR1-CD8TM-CD28-OX40-ζ (SEQ. ID. No. 5).

FIG. 19 shows the amino acid sequence of human MR1-CD8-CD28TM-OX40-ζ (SEQ. ID. No. 8) and the nucleic acid encoding MR1-CD8-CD28TM-OX40-ζ (SEQ. ID. No. 7).

FIG. 20 shows the amino acid sequence of human MR1-1-CD8-ζ (SEQ. ID. No. 10) and the nucleic acid encoding MR1-1-CD8-ζ (SEQ. ID. No. 9).

FIG. 21 shows the amino acid sequence of human MR1-1-CD28-ζ (SEQ ID No. 12) and the nucleic acid encoding MR1-1-CD28-ζ (SEQ. ID. No. 11)

FIG. 22 shows the amino acid sequence of human MR1-1-CD8TM-CD28-OX40-ζ (SEQ. ID. No. 14) and the nucleic acid encoding MR1-1-CD8TM-CD28-OX40-ζ (SEQ. ID. No. 13).

FIG. 23 shows the amino acid sequence of human MR1-1-CD8-CD28TM-OX40-ζ (SEQ. ID. No. 16) and the nucleic acid encoding MR1-1-CD8-CD28TM-OX40-ζ (SEQ. ID. No. 15).

### DETAILED DESCRIPTION OF THE INVENTION

Chimeric immunoreceptor (CIR) technology (see review by Kershaw 2005) is based on the recognition that it is possible to endow new targeting and functional specificities on T-cells (and other cell types, e.g., natural killer (NK) cells) through gene transfer of chimeric nucleic acids that encodes fusion proteins comprising an extracellular binding domain with the signaling motifs attached to the endogenous T-cell receptor (TCR). This direct coupling bypasses the need for MHC (major histocompatibility complex) presentation of antigen and can permit direct zeta chain dimerization and intracellular signaling in the absence of MHC presentation of antigen. This strategy is especially useful in treating brain cancer where MHC protein expression in the brain is absent under normal circumstances and also is absent in many very aggressive tumors.

CIR technology offers a strategy for targeting invasive tumor cells that express an antigen of interest. CIRs have been used in a number of preclinical cancer models (Hwu et al. 1995; Altenschmidt et al. 1997) and have also been utilized in a clinical HIV trial by creating T-cells designed to target and destroy HIV-1 infected cells in Phase I/II studies (Mitsuyasu et al. 2000; Deeks et al. 2002). Other Phase I studies are currently under way using this technology (Lamers et al. 2002; Wang et al. 2004; Kahlon et al, 2004). Adoptive immunotherapy targeting common glioma antigens such as EGFRvIII, a mutant variant form of the epidermal growth factor receptor, with an engineered cell that will target glioma cells for killing, provides an important strategy for attacking the widely disseminated glioma cells in the brain.

EGFRvIII is a mutant variant form of the epidermal growth factor receptor. This mutant is found only or primarily on the surface of glioblastoma cells, and on cells of breast, ovarian, non-small cell lung carcinomas, head and neck squamous cell carcinoma, and bladder carcinoma. EGFR upregulation has negative prognostic significance in GBM (Shinojima et al, 2003) and patients with tumor cells that express the vIII mutant have an even worse prognosis. EGFRvIII is formed by an 801 by in-frame deletion in the extracellular binding domain of the epidermal growth factor receptor. The vIII mutant does not bind EGF but is able to self-dimerize and phosphorylate ERBB-2 in a ligand independent fashion (Tang et al, 2000; Fernandes et al, 2000). The deletion results in a constitutively active tyrosine kinase activity in the absence of any ligand EGF, giving rise to the aberrant EGFR signaling. The mutant EGFRvIII is detected in a majority of glioblastoma specimens (Moscatello et al, 1995). Furthermore, EGFRvIII expression is correlated with the invasiveness of glioblastoma tumors (Lal et al, 2002). Finally, it has not been detected in normal tissues, potentially providing an improved safety profile over strategies targeting the overexpressed wild-type receptor. The perturbation of EGFR expression and the expression of the ligand independent oncogenic vIII variant are important targets for glioma therapy.

The use of anti-EGFR antibodies to target tumor cells overexpressing wild type and mutant EGFR has been reported (Modjtahedi, et. al., 2003). Anti-EGFR antibodies made against the wild type EGFR can also bind the mutant EGFRvIII. However, although these anti-EGFR antibodies were potent inhibitors of cell growth of cells expressing the wild type EGFR, the antibodies did not directly inhibit the growth of EGFRvIII expressing cells nor the constitutive tyrosine kinase activity of this receptor in in vitro experiments.

CIR-based technology using antibody as the targeting moiety in CIR has been reported for a wide range of target molecules, including HIV gp120 (Tran, 1995), CD20 (Jensen, 1998), VEGF (Niederman, 2002), CEA (Gilham, 2002; Hombach, 1999) and TAG-72 (McGuinness, 1999). The structures of these CIRs are varied. Mere direct substitution of targeting moiety among these CIRs does not necessarily produce T cells with optimally functional CIRs. In one study comparing the targeting and cytotoxic activities of four different CIRs, each having a different antigen binding domain of a single chain binding antibody, scFv, as the targeting moeity, two CIRs (CIRs against oncofetal antigen 5T4 and neural cell adhesion molecule) required an extracellular spacer region between the scFv region and the transmembrane region for enhanced specific cytotoxic activity. On the other hand, the remaining two CIRs (CIRs against carcinoembryonic antigen CEA and B-cell antigen CD19) displayed optimal cytotoxic activity only in the absence of an extracellular spacer region (Guest, 2005). Thus, the fusion of targeting moieties to endogenous T-cell receptor signaling domains is unpredictable as to whether or not a functional protein will be expressed and whether the CIR confers cytotoxic activity to the T-cell expressing the CIR. Other difficulties with CIR technology include unpredictability with the CIR's protein expression, protein export and presentation extracellularly, interaction with target ligand, proliferation of the T-cells expressing the CIRs, and the activation of the T-cell expressing the CIRs without the benefit of MHC presenting cells (Kershaw, 2005). In addition, the level of expression of the chimeric T-cell receptor, the binding affinity of the chimeric receptor to the target antigen, and the level of antigenic protein expression on the tumor cell surface may influence in unpredictable ways the level of cytotoxic activity of the T-cells expressing the chimeric receptor (Turatti F, 2007). Accordingly, among the scientific community, there have been several unsuccessful attempts at constructing functional CIRs specially targeting EGFRvIII (personal communications).

The present invention is based on the discovery that a humanized chimeric protein, a CIR, MR1-CD8-ζ (SEQ. ID. No:2), when introduced by gene transfer of a nucleic acid (SEQ. ID. No: 1) into human T-cells, redirects the T cell's lytic capacity to kill EGFRvIII expressing tumor cells. The MR1-CD8-ζ is expressed on the cell surface of the transfected human T-cell. Herein is described long-term expression, specific lysis of target cells, and secretion of pro-inflammatory cytokines upon engagement of this receptor.

As used herein, the term “humanized” refers to a non-human protein having a part of the complete protein sequence substituted with human protein sequence. For example, a humanized mouse T-cell receptor can have the mouse TM and intracellular signaling domain substituted with a human T-cell receptor protein sequence corresponding to the TM and intracellular signaling domain.

As used herein, the term “chimeric” describes being composed of parts of different proteins or DNAs from different origins. For example, the humanized chimeric protein, MR1-CD8-ζ (SEQ. ID No:2) comprises several polypeptide portions: (a) an antigen binding portion, MR1scFv, of MR1 single chain antibody, (b) a human CD8α hinge and transmembrane portion, and (c) a human T-cell receptor zeta chain (TCR ζ) intracellular signaling portion. The antigen binding portion, MR1 scFv, binds specifically to the EGFRvIII, thus facilitating targeting of the MR1-CD8-ζ expressing T-cells to EGFRvIII expressing cells such as in gliomablastoma, the breasts, and the lungs. The different protein portions: mouse MR1scFv, a human CD8α hinge and transmembrane portion, and a human TCR ζ intracellular signaling portion, are derived from different parent proteins and they are arranged from the amino to the carboxyl terminus as set forth herein in the single humanized chimeric polypeptide. A nucleic acid encoding the humanized chimeric protein, MR1-CD8-ζ, is provided in SEQ. ID. No:1 and it is a chimeric nucleic acid comprising nucleic acid sequences of different coding sequences: coding sequences of mouse MR1scFv, a human CD8α hinge and transmembrane portion, and a TCR ζ intracellular signaling portion.

The term “coding sequence” means the nucleic acid sequence which is transcribed (DNA) and translated (mRNA) into a polypeptide in vitro or in vivo when operably linked to appropriate regulatory sequences.

The intracellular domains can contain the signaling domains of proteins. As used herein, signaling domain refers to the part of the protein that participates in transducing the message of effective ligand binding into the interior of the T-cell to elicit cytotoxic activity in the cell, such as the release of cytotoxic factors to the ligand-bound target cell, or other cellular responses elicited with ligand binding.

In the one embodiment, the chimeric protein MR1-CD8-ζ is a humanized CIR, containing all human-derived sequences except for the MR1 scFv antigen binding region, which is derived from a mouse single chain antibody. The extensive humanization of a chimeric protein helps to reduce eliciting immune response to the chimeric protein in the human host.

In one embodiment, the humanized chimeric proteins of the invention comprise three protein portions: (1) an extracellular domain comprising an EGFRvIII binding portion, serving to target the humanized chimeric protein expressing T-cell to a EGFRvIII expressing cell; (2) a middle portion comprising a transmembrane domain (TM) derived from a human protein for anchoring the humanized chimeric protein to a T-cell expressing the chimeric protein. The middle portion also serves in part to connect the extracellular domain to the intracellular domain. The middle portion can also serve to extend the extracellular EGFRvIII binding domain away from the T-cell plasma membrane so that the extracellular EGFRvIII binding domain can interact with a target EGFRvIII. The second portion can also assist in the intracellular signaling of the T-cell following the binding of the extracellular EGFRvIII-binding domain, thus relaying the message of EGFRvIII binding from the exterior of the T-cell to the interior of the T-cell; and (3) an intracellular domain comprising the human T-cell receptor zeta chain (TCR ζ, also known as CD3ζ).

In one embodiment, the extracellular EGFRvIII binding domain of the humanized chimeric proteins described herein comprises an antigen binding domain of the mouse single chain antibody specific for EGFRvIII, MR1. The antigen for MR1 is EGFRvIII. The antigen binding region of MR1 comprises the variable fragment of the MR1, MR1scFv. In another embodiment, an antigen binding domain of MR1scFv comprise amino acid mutations ST→PY (at amino acids 98 and 99 of the V_{H} of MR1scFv) and F→V (at amino acid 92 of the V_{L} of MR1scFv), which is MR1-1 scFv. MR1-1 scFv has a higher binding affinity for the antigen EGFRVIII than its parent MR1scFv (Beers, Clin. Can. Res. 2000, 6:2835-43; Kuan, Clin. Can. Res. 2000, 88:962-9). In one embodiment, the antigen binding region of MR1 comprises the V_{H} or V_{L} single domain variable fragment of MR1scFv or MR1-1scFv, the.

In one embodiment, the middle portion of the humanized chimeric proteins described herein comprises a human CD8α hinge and TM. In another embodiment, the middle portion of the humanized chimeric proteins described herein comprises a human CD28 protein fragment comprising an extracellular domain, a TM, and an intracellular domain. In another embodiment, the middle portion of the humanized chimeric proteins described herein comprises a fusion protein comprising a human CD8α hinge and TM, a human CD28 intracellular domain and a human OX40 intracellular domain. In yet another embodiment, the middle portion of the humanized chimeric proteins described herein comprises a fusion protein comprising a human CD8α hinge, a TM and an intracellular signaling domain of a human CD28, and an intracellular signaling domain of a human OX40.

As set forth herein, the combinations of the extracellular EGFRvIII binding domain, the middle portion and the intracellular domain provide several similar humanized chimeric proteins that can target T-cells to EGFRvIII expressing cells. In one series of humanized chimeric proteins, the MR1scFv antigen binding domain is replaced by the higher affinity EGFRvIII binding domain MR1-1. MR1-1scFv is derived from MR1scFv through two mutations in the CDR3 region of the V_{H} and V_{L} chains (Beers, 2000; and Kuan, 2000 supra; and WO/2001/062931). In one series of humanized chimeric proteins described herein, the extracellular EGFRvIII binding region comprises the V_{H} single domain of MR1svFc. In another series of humanized chimeric proteins of this invention, the extracellular EGFRvIII binding region comprises the V_{L} single domain of MR1svFc. In yet another series, the extracellular EGFRvIII binding region comprises the V_{H} single domain of MR1-1svFc. In another series, the extracellular EGFRvIII binding region comprises the V_{H} single domain of MR1-1svFc.

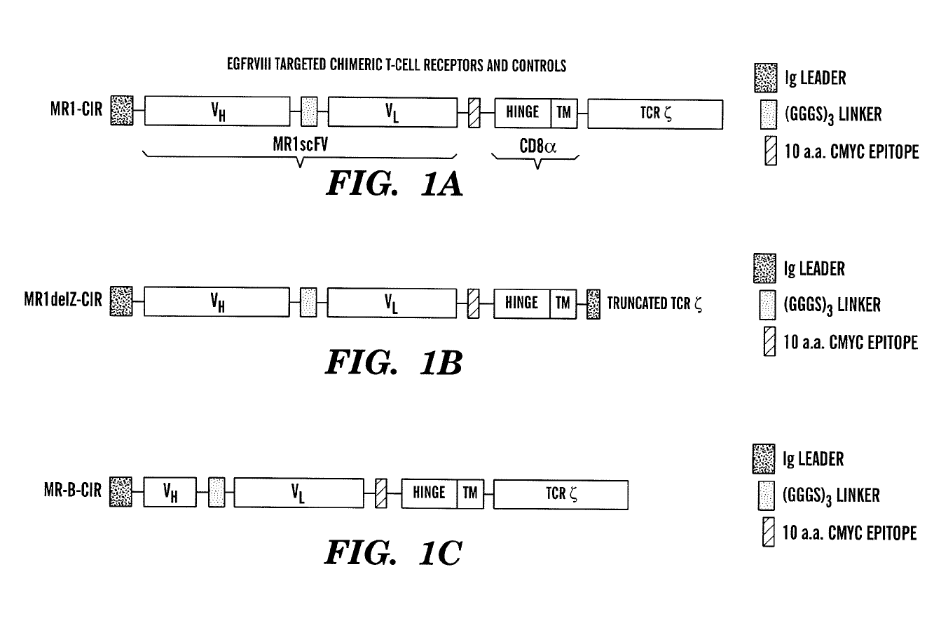

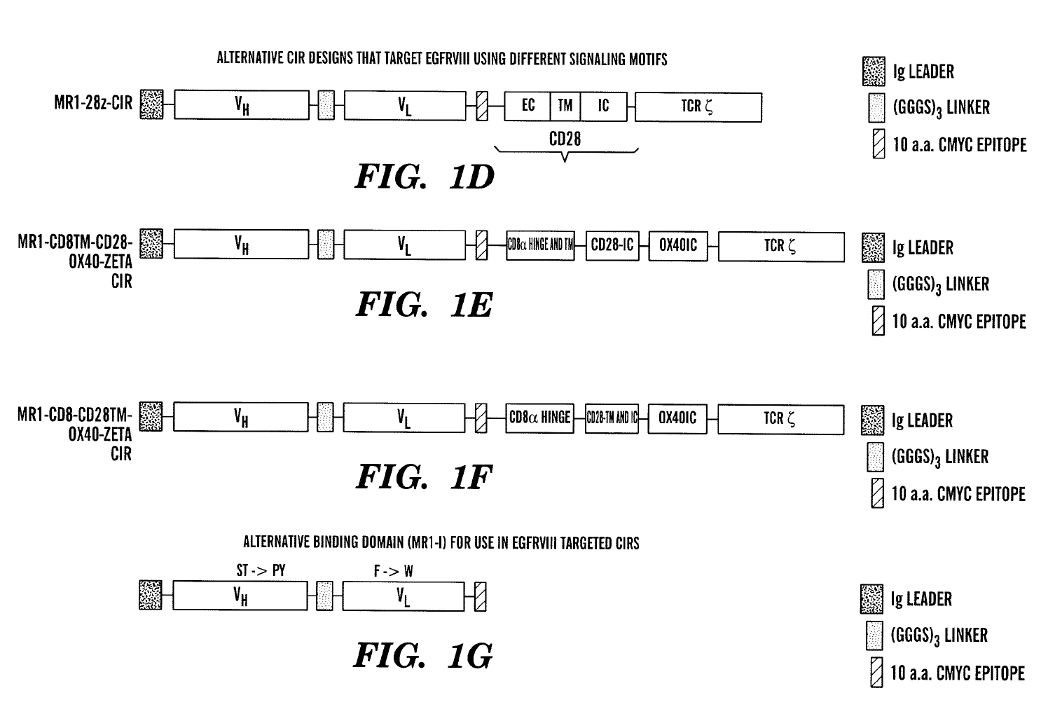

In another modification of the humanized chimeric protein MR1-CD8-ζ, the signaling domains of the chimeric receptors have been replaced with dual or tripartite signaling motifs (FIG. 1E and FIG. 1F), for example, the intracellular signaling portions of the human CD28 and OX40. The inclusion of the tripartite signaling motif can enhance the in vitro and in vivo efficacy of human T-cells expressing these receptors.

Encompassed in the invention are nucleic acids comprising the coding sequences of the humanized chimeric proteins described herein comprising an extracellular EGFRvIII binding domain, a middle portion comprising a TM derived from a human protein, and an intracellular domain disclosed herein. The nucleic acids are SEQ. ID. Nos. 1, 3, 5, 7, 9, 11, 13, and 15.

Also encompassed in the invention are nucleic acid constructs comprising the coding sequences of the humanized chimeric proteins described herein comprising an extracellular EGFRvIII binding domain, a middle portion comprising a TM derived from a human protein, and an intracellular domain disclosed herein. Such nucleic acid constructs are meant for the replication and/or expression of the coding sequences of the humanized chimeric proteins described herein. The nucleic acid constructs can comprise replication and/or expression regulatory elements such as 5′ upstream and 3′ downstream regulatory elements such as promoter sequences, ribosome recognition and binding TATA box, and 3′ UTR AAUAAA transcription termination sequence for the efficient gene transcription and translation in the transfected T-cell. As a nucleic acid construct for expression, also termed an expression vector, the construct can have additional sequence such as 6X-histidine, V5, thioredoxin, glutathione-S-transferase, c-Myc, VSV-G, HSV, and FLAG tag which are incorporated into the expressed humanized chimeric protein for identification and detection purposes in vivo.

In one embodiment, the invention provides chimeric EGFRvIII targeting T cell receptors encoded by a nucleic acid comprising the coding sequences of the humanized chimeric proteins described herein comprising an extracellular EGFRvIII binding domain, a middle portion comprising a TM derived from a human protein, and an intracellular domain disclosed herein. In one embodiment, the chimeric EGFRvIII targeting T cell receptor has the sequence of SEQ. ID. No. 2, 4, 6, 10, 12, 14 or 16.

Also encompassed in the invention are cells comprising the nucleic acid constructs comprising the coding sequences of the humanized chimeric proteins comprising an extracellular EGFRvIII binding domain, a middle portion comprising a TM derived from a human protein, and an intracellular domain disclosed herein. In one embodiment, the cells are T-cells. In another embodiment, the cells are human cells. In yet another embodiment, the cells are mammalian cells. These cells express the humanized chimeric proteins described herein on the cell surface.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987)).

Chimeric T-Cell Receptor Design and Construction

Conventional polymerase chain reaction (PCR) cloning techniques can be used to construct an isolated chimeric nucleic acid encoding the humanized chimeric proteins disclosed herein. The isolated chimeric nucleic acid can be cloned into a general purpose cloning vector such as pUC19, pBR322, pBluescript vectors (Stratagene Inc.) or pCR TOPO® from Invitrogen Inc. The resultant nucleic acid construct (recombinant vector) carrying the isolated chimeric nucleic acid encoding a humanized chimeric protein disclosed herein can then be subcloned into expression vectors or viral vectors for protein expression in mammalian cells. The mammalian cells are preferably human T-cells.

Several different chimeric T-cell receptors were designed as shown in FIGS. 1A-1G to target human T-cells to EGFRvIII. There are two different potential binding motifs to use to target the EGFRvIII protein, MR1 and MR1-1. FIGS. 1A-1F show a schematic of receptors that are based on MR1, a single chain antibody which binds to EGFRvIII. FIG. 1G shows the 3 amino acid differences in MR1-1 as compared to MR1, as described by Beers et al. 2000. Each construct was ligated into the multiple cloning site of the mammalian plasmid expression vector pMG (Invitrogen, San Diego, Calif.) under the control of the human Elongation Factor 1α promoter (EF1p).

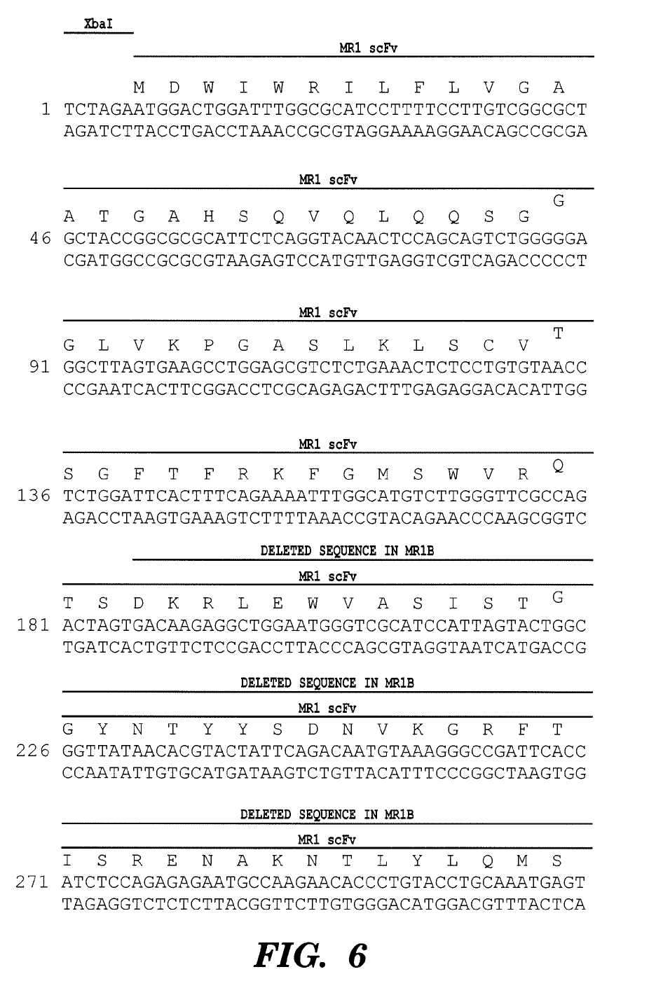

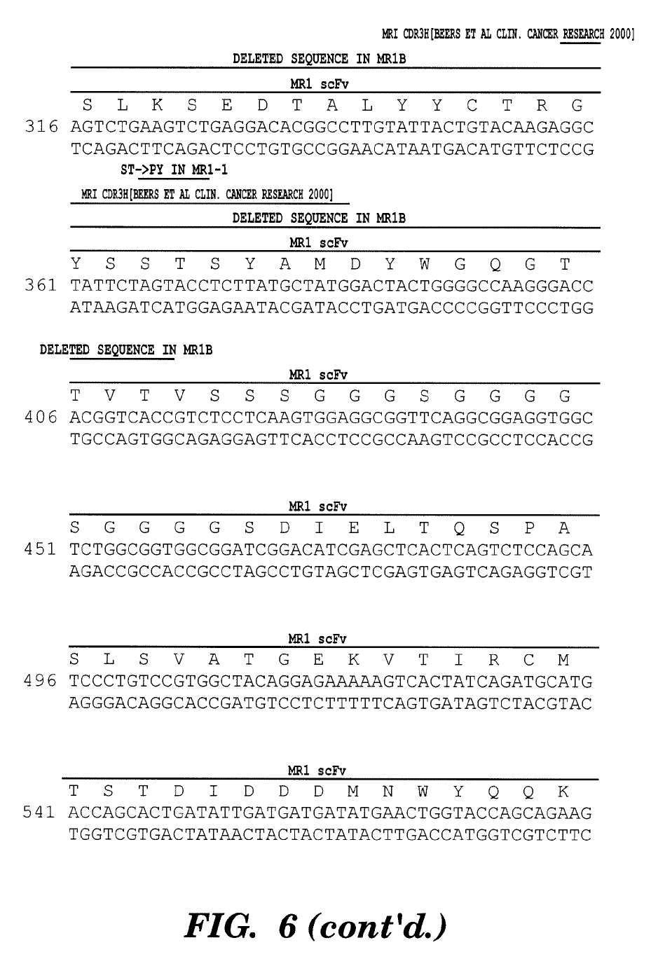

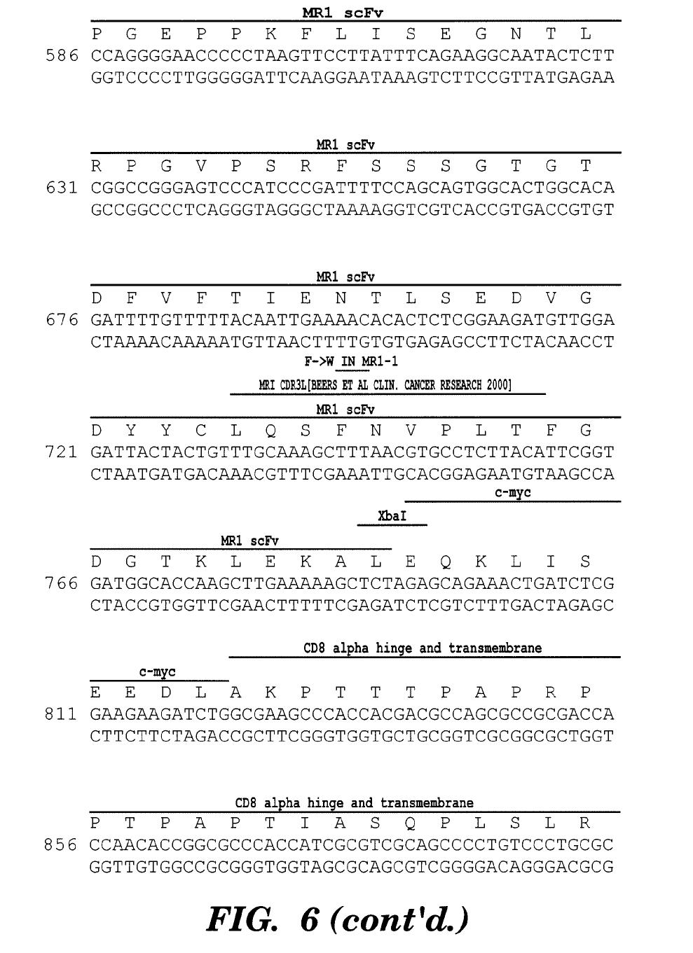

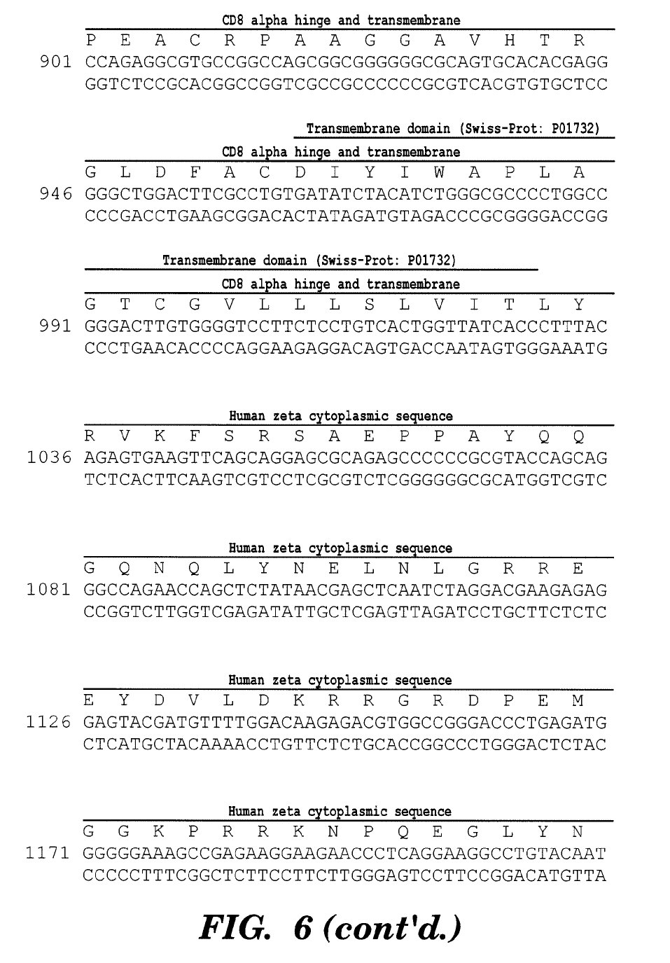

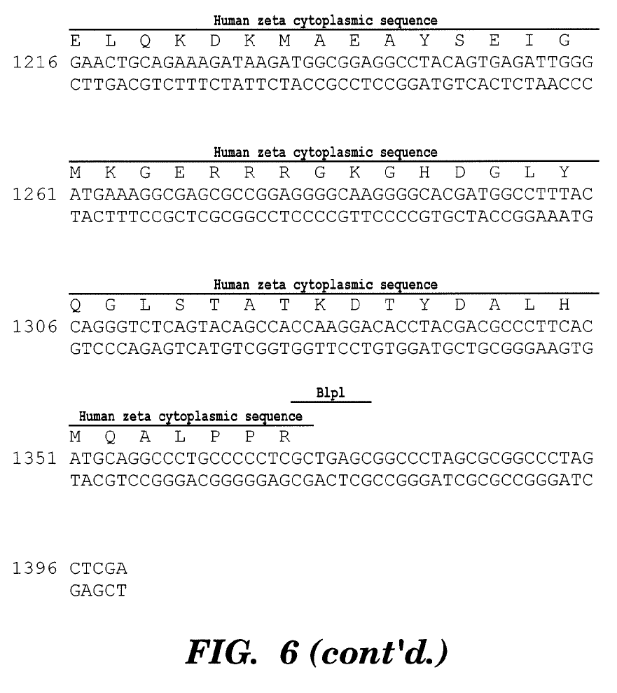

The initial testing of this concept was performed with the CIR shown in FIG. 1A, the MR1-CD8-ζ-CIR. MR1-CD8-ζ (FIG. 1A) chimeric T-cell receptor (cTCR) consists of a single chain antibody variable (scFv) binding domain known as MR1 (Lorimer et al, 1996), linked to the hinge and TM of human CD8α, which is fused to the intracellular signaling domain of the human CD3ζ chain. MR1 binds EGFRvIII directly, but it does not recognize the wild-type EGFR. The DNA sequence and encoded amino acid sequence of this construct are shown in FIG. 6.

MR1-CD8-Delζ (FIG. 1B) contains a truncated version of the cytoplasmic zeta chain. Three hundred base pairs were eliminated of the 336 base pair long zeta chain and a TGA stop codon was substituted for the first intracellular TAC codon (Y) as described earlier for the zeta chain (Niederman et al. 2002).

MR1-B-CD8-ζ (FIG. 1C), a binding mutant version MR1-CD8-ζ, was created by a 228 base pair deletion in the heavy chain including the 33 base pair heavy chain complentarity determining region 3 (VHCDR3) which plays a crucial role in binding of MR1 to EGFRvIII (Beers et al. 2000).

Each cTCR also contained a c-myc epitope tag (EIKLISEED) (SEQ. ID. No:17) or (EDKLISEED) (SEQ. ID. No: 27) to enable easy detection. Vectors were produced by fusion polymerase chain reaction (PCR) and were ligated into a pMG expression vector that was modified to coexpress the Hygromycin phosphotransferase-HSV thymidine kinase (HyTK) selection/suicide fusion gene (Lupton S D et al. Mol Cell Biol 1991). The pMG-HyTK expression vector was a generous gift of Dr. Michael C. Jensen (City of Hope National Medical Center, Duarte, Calif.). Transcription of the chimeric construct is driven by a modified human Elongation Factor-1α (EF1α) promoter whereas the expression of HyTK fusion-protein is controlled by human cytomegalovirus (CMV) promoter.

Several other vectors have been designed with alternative signaling domains as shown in FIGS. 1D-1F. These incorporate additional signaling motifs beyond those of TCR-ζ and include CD28 and OX40 signaling motifs, that have been shown to provide additional co-stimulatory signals to the chimeric receptor. These constructions, as shown, are examples of alternative signaling domains that can be used to direct human T-cells to EGFRvIII expressing tumors.

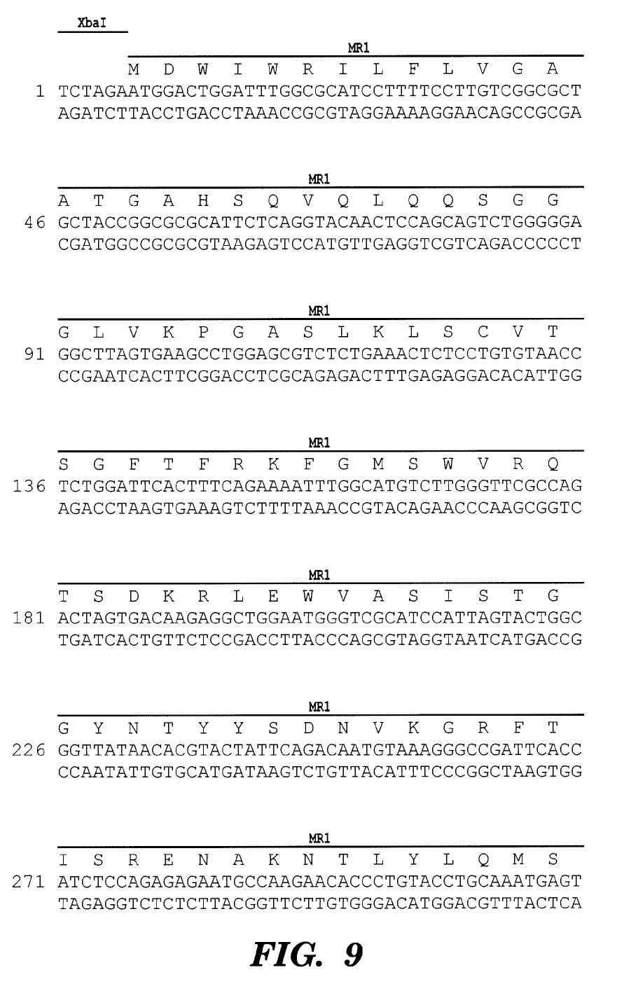

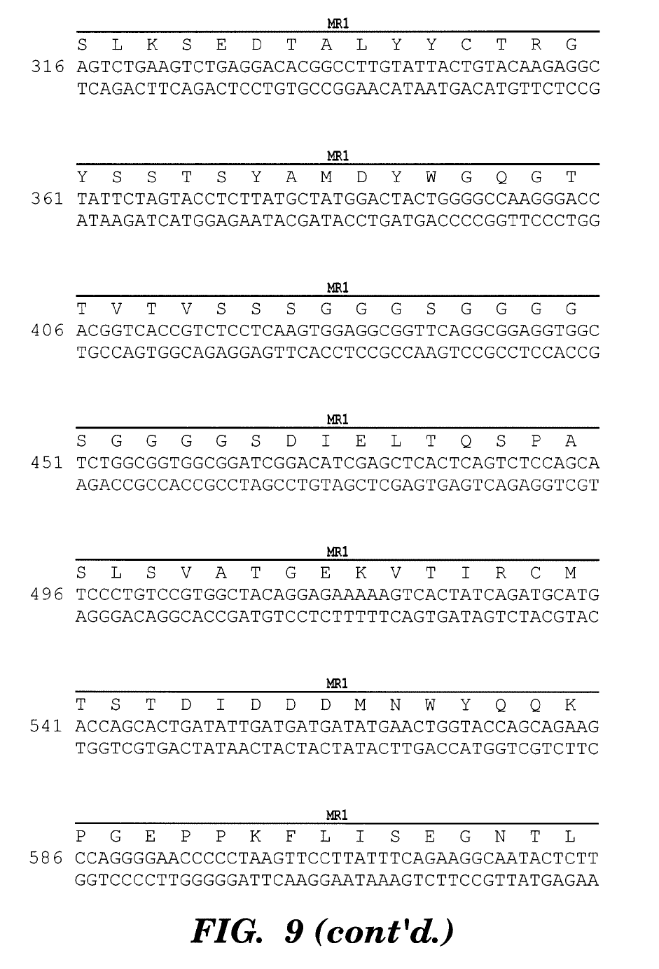

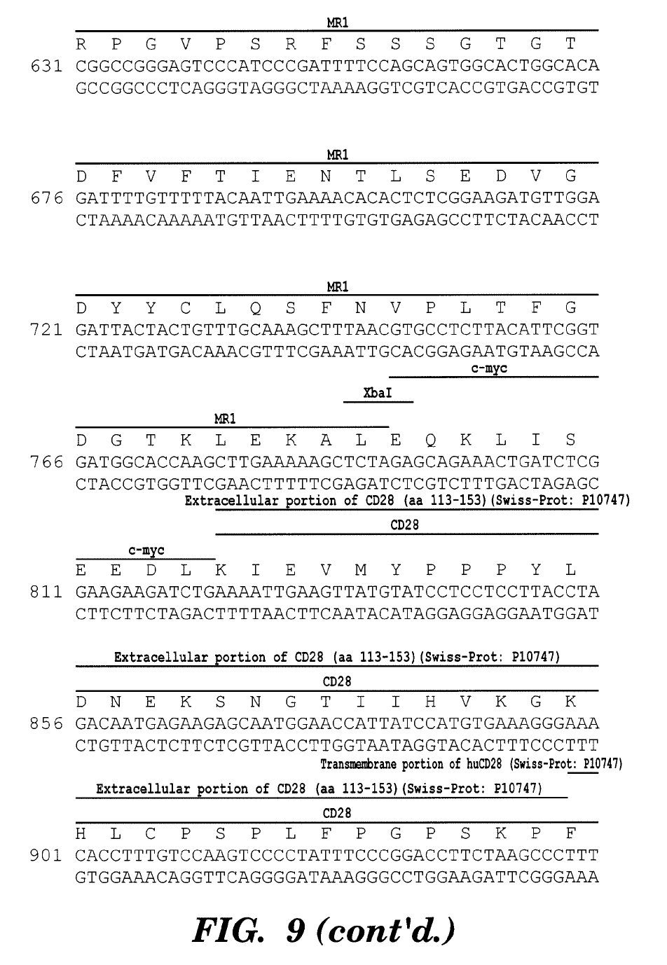

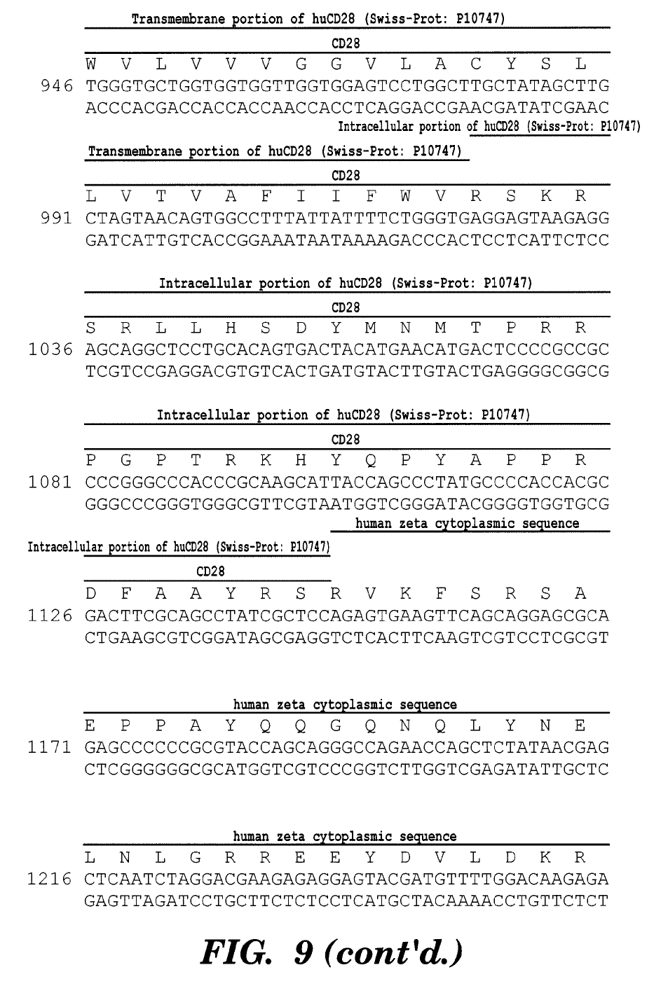

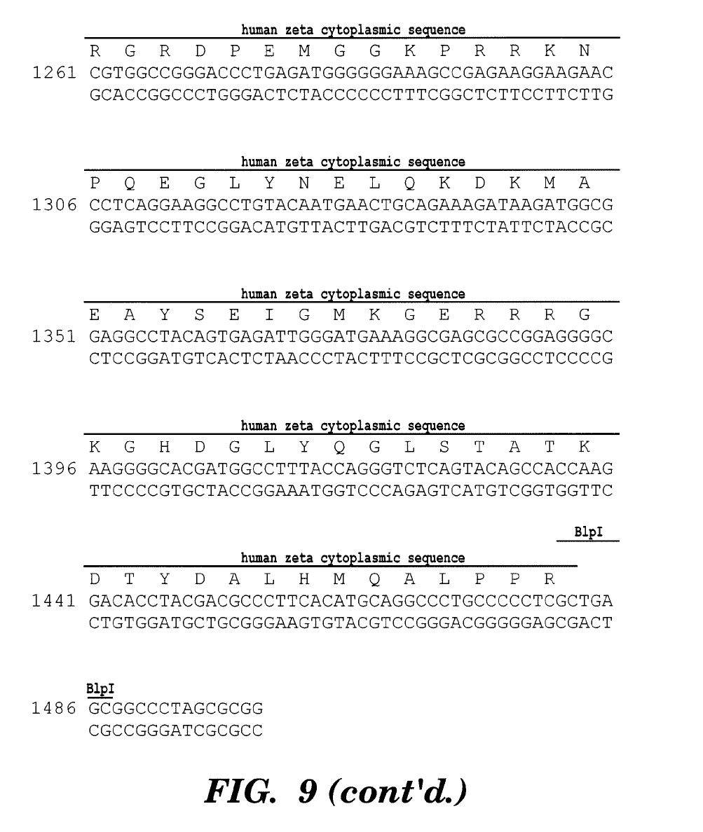

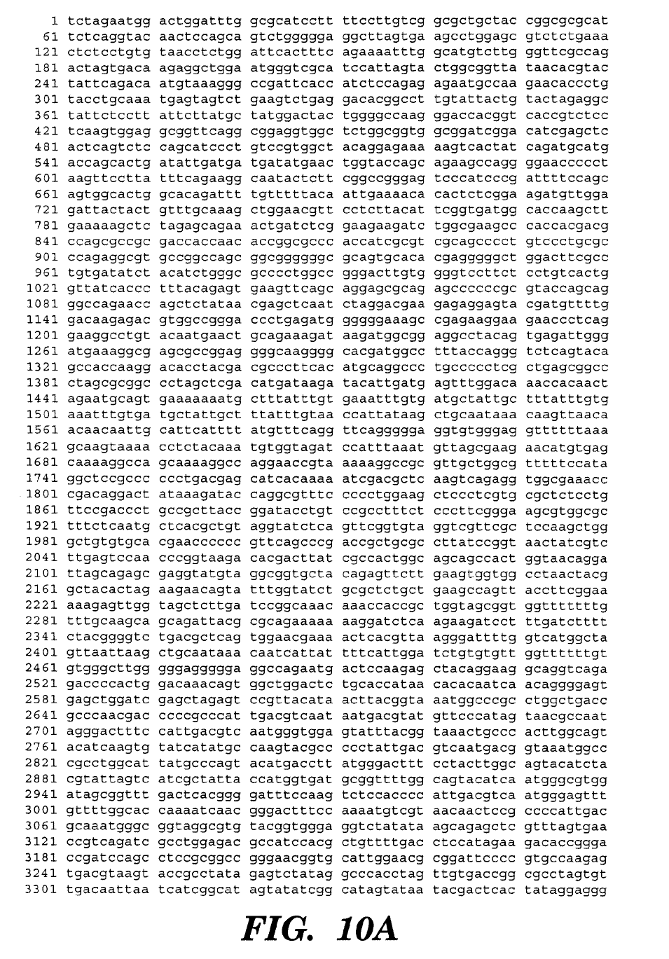

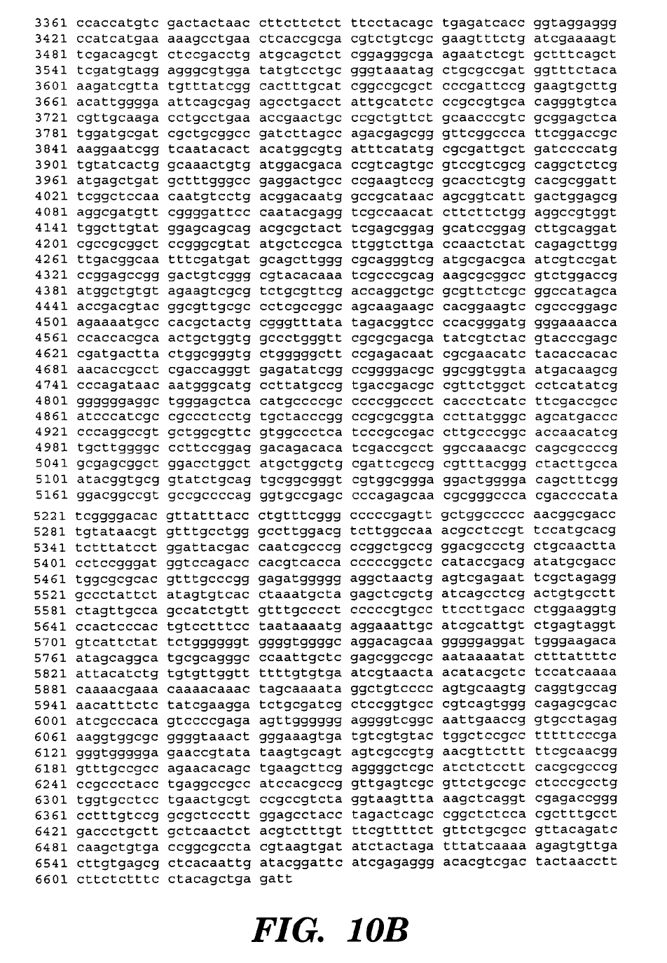

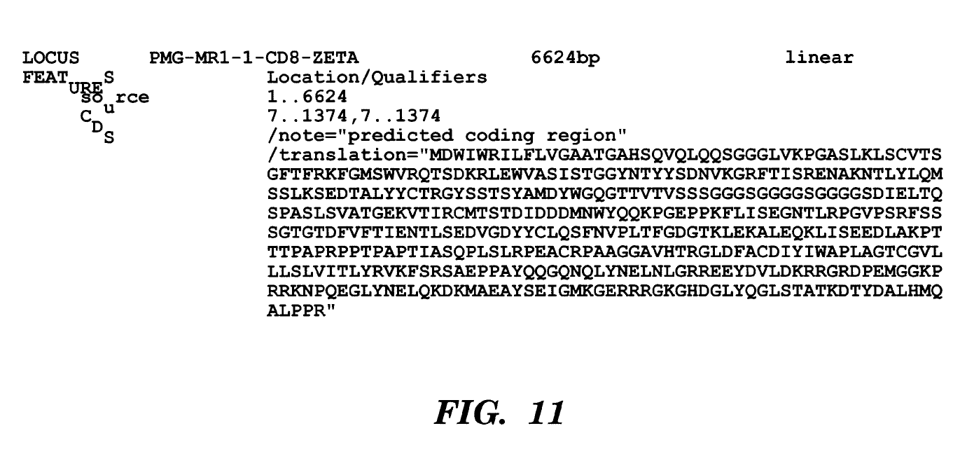

MR1-CD28-ζ (FIG. 1D) shows that the MR1 scFv is coupled to the human CD28 gene in its entirety fused directly to the intracellular signaling domain of human TCRζ. The DNA sequence and encoded amino acid sequence of this construct are shown in FIG. 9.

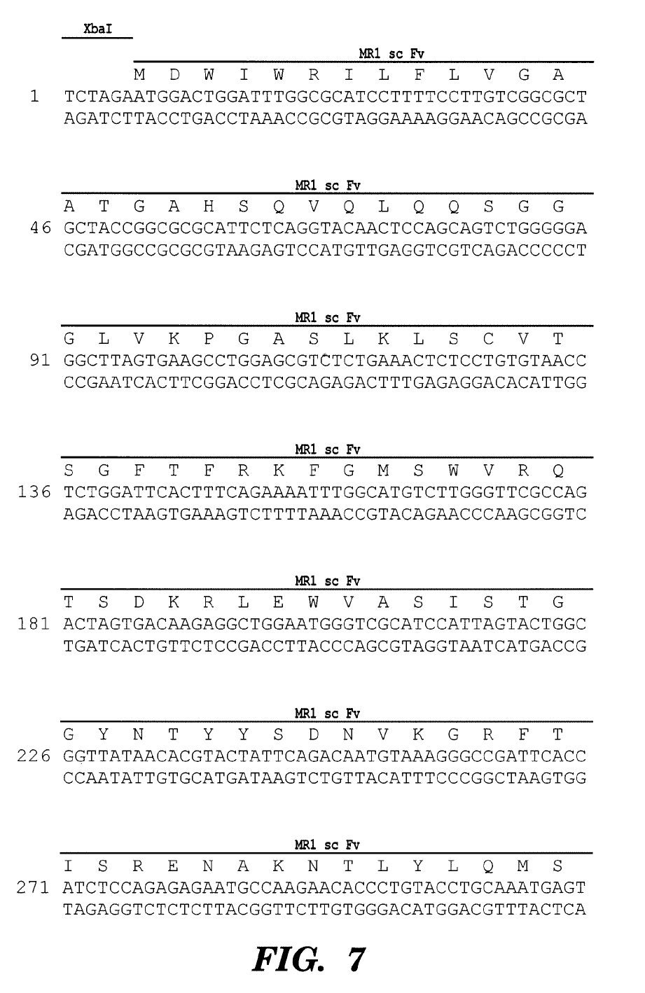

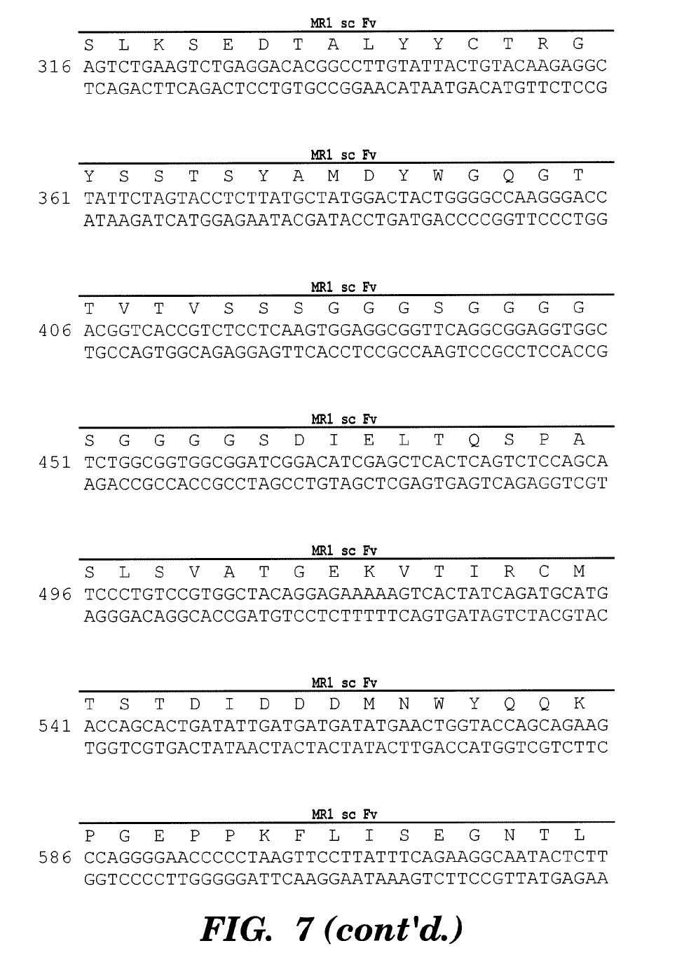

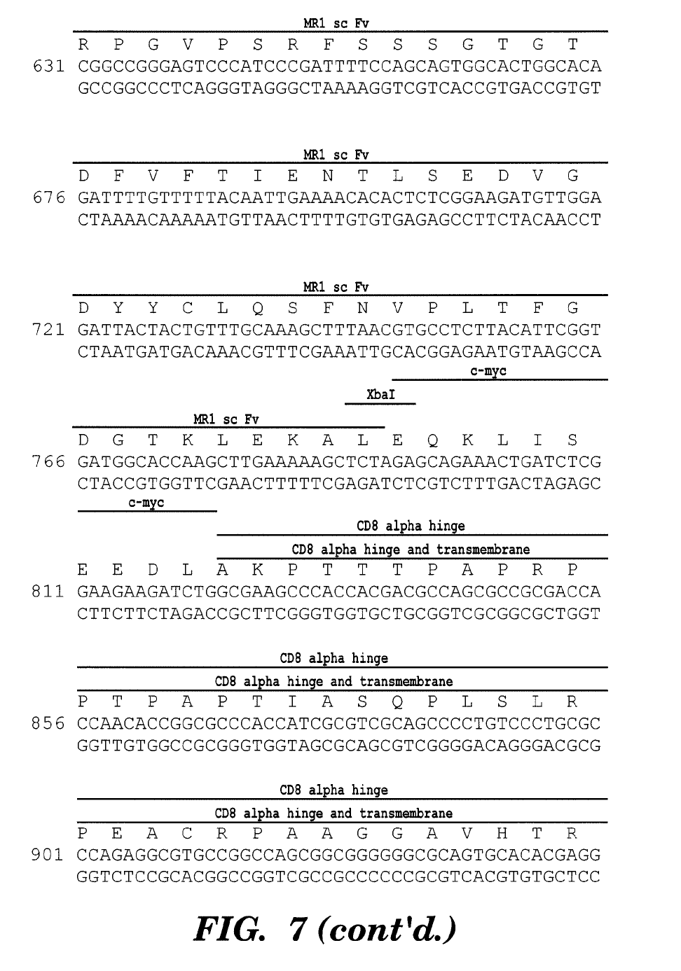

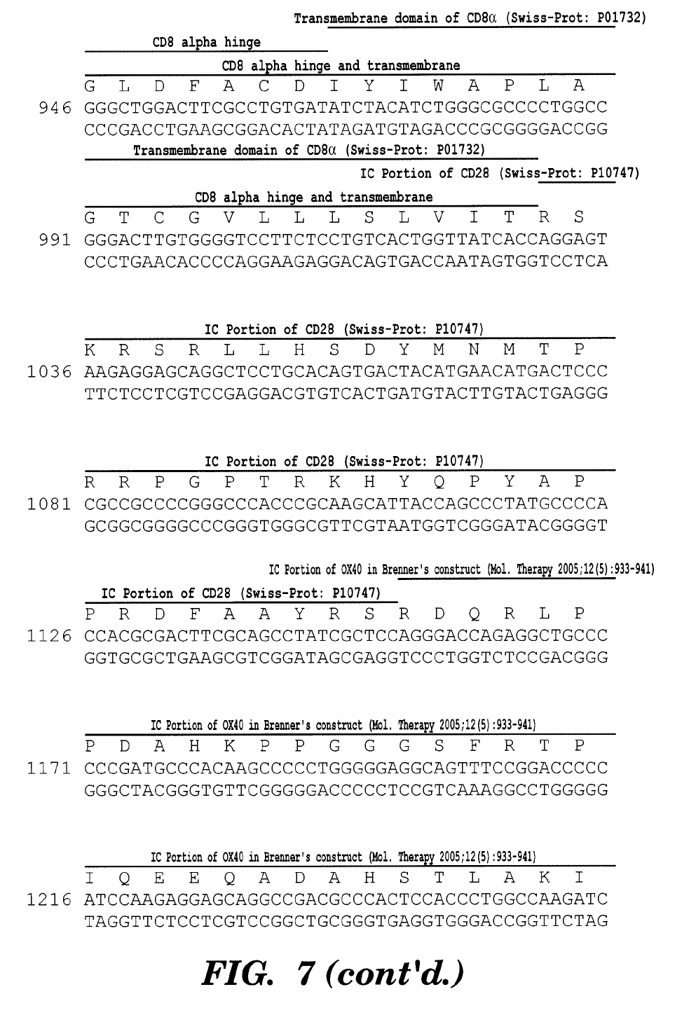

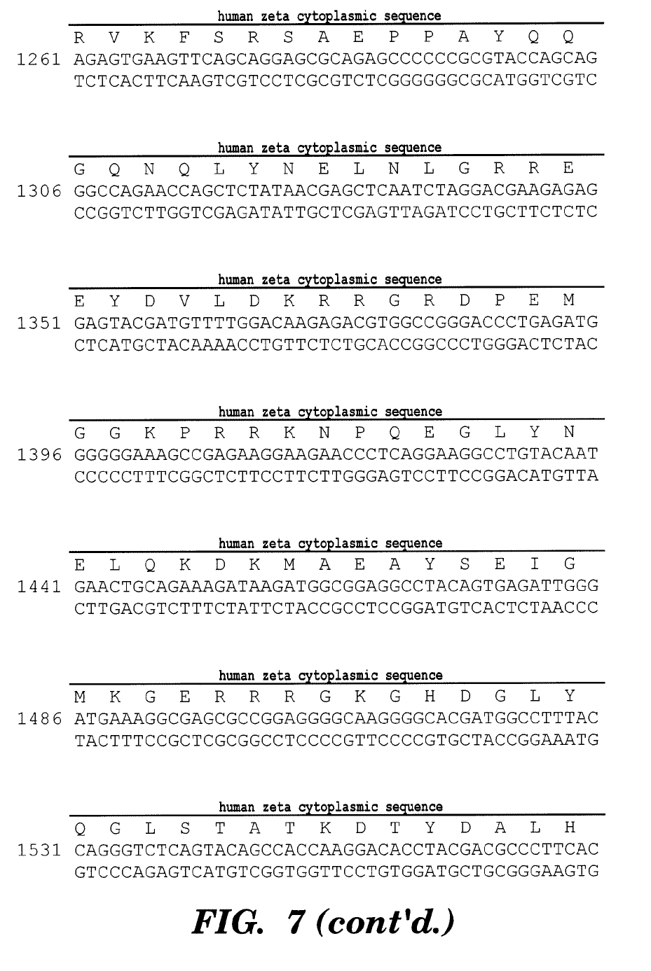

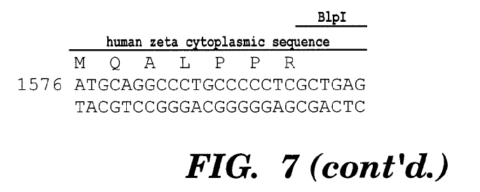

MR1-CD8TM-CD28-OX40-ζ (FIG. 1E) shows a coupling of the MR1scFv to the CD8α hinge region and TM, the CD28 intracellular domains, the OX40 intracellular signaling domain, and the human TCR ζ. This is known as a tripartite signaling construct, as it contains signaling domains from three different proteins (CD28, OX40, TCRζ) and, as described by Pule et al. (Pule, M. A., et al., Mol. Ther. 12(5): 933-941, 2005) can improve co-stimulatory signaling of an EGFRvIII directed human T-cell. The amino acid sequence and DNA sequence of this construct are shown in FIG. 7.

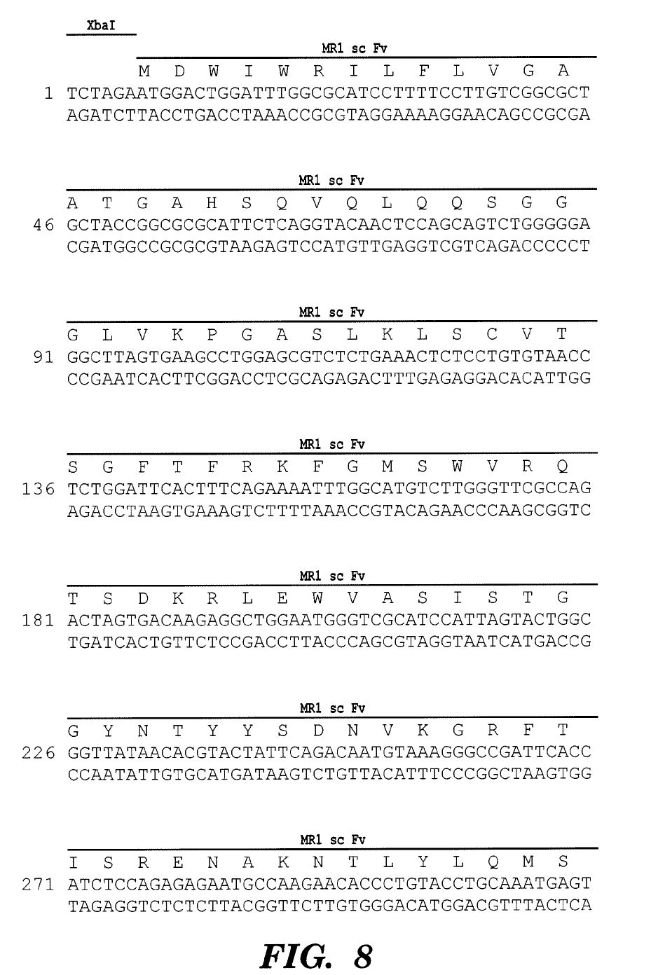

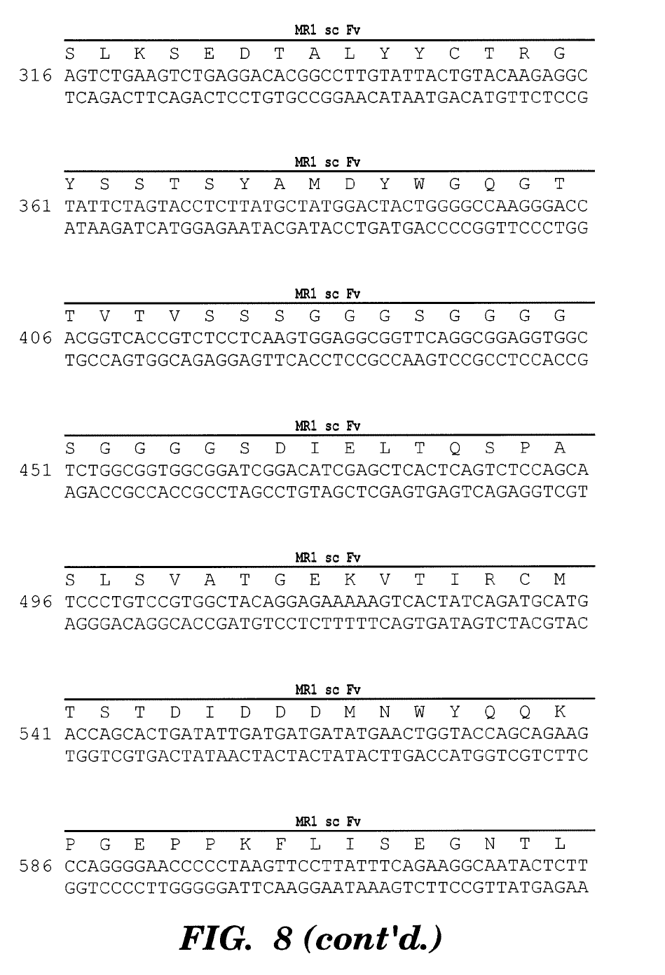

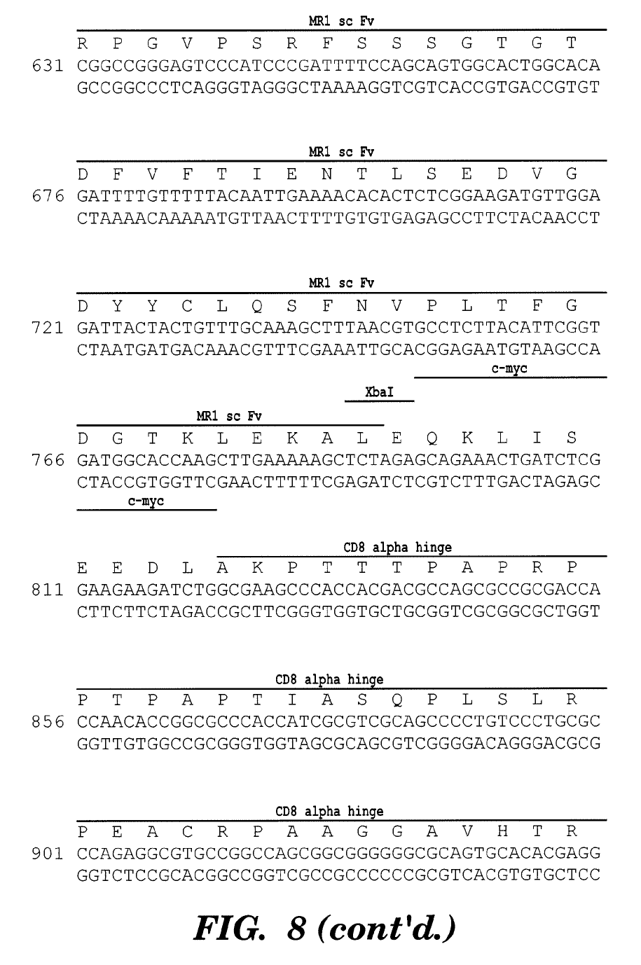

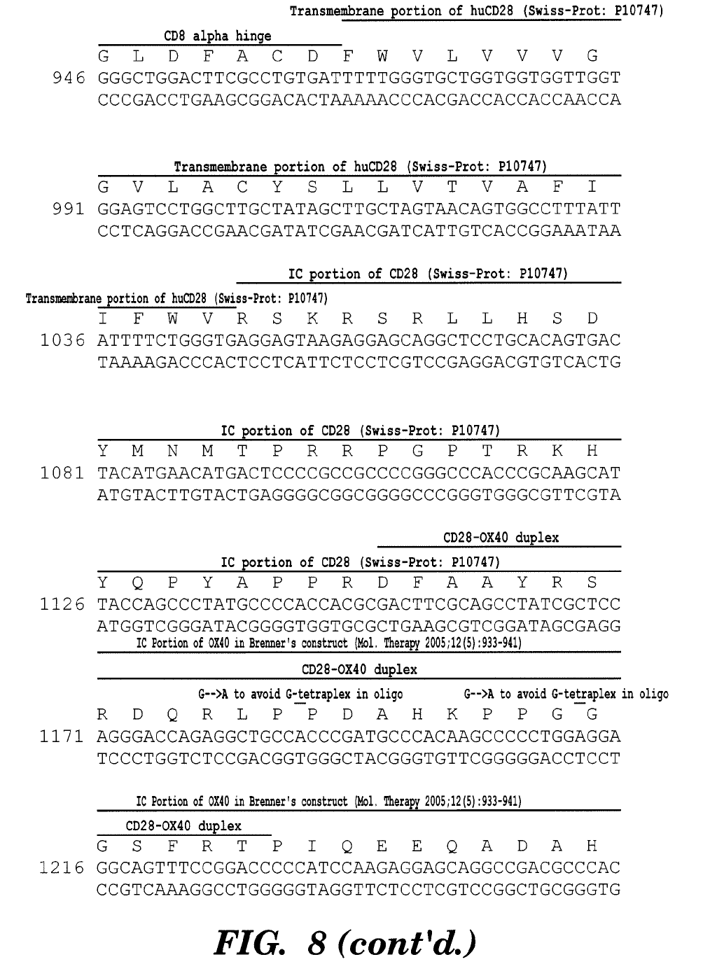

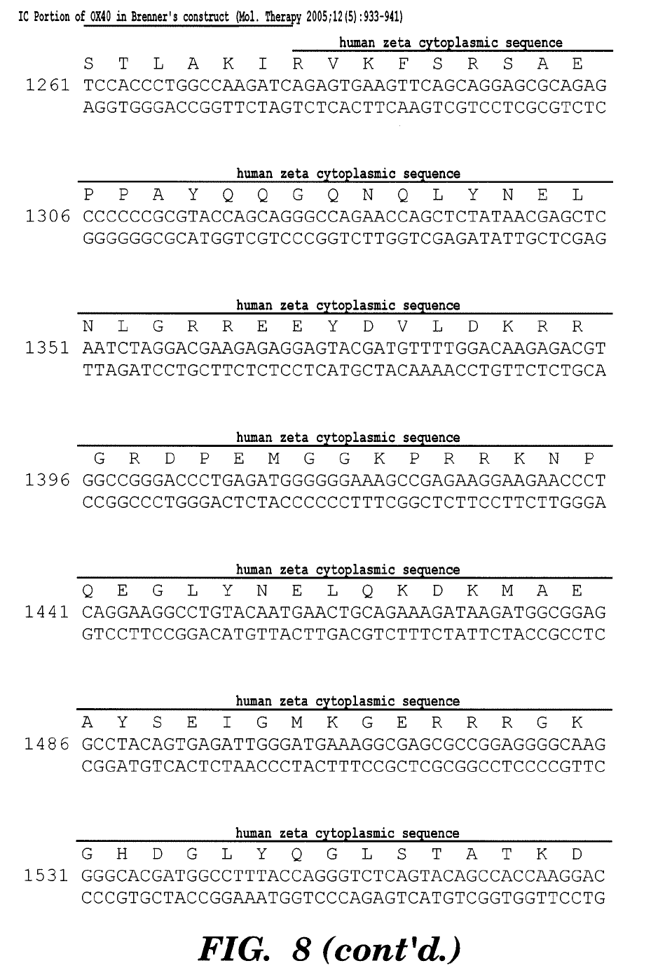

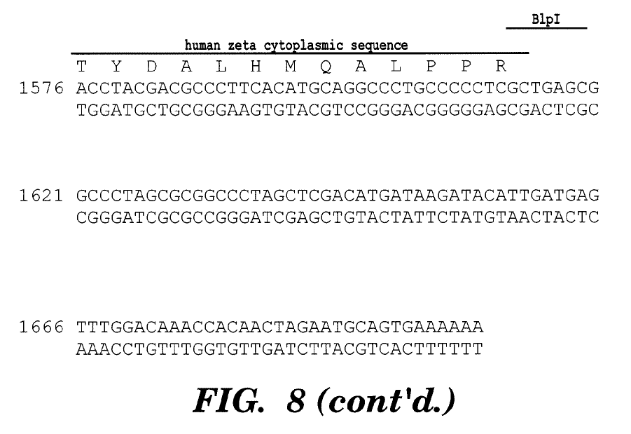

MR1-CD8-CD28TM-OX40-ζ (FIG. 1F) shows a coupling of the MR1scFv to the CD8α hinge region, the CD28 transmembrane and intracellular domains, the OX40 intracellular signaling domain, and the human TCR ζ. The amino acid sequence and DNA sequence of this construct are shown in FIG. 8.

MR1-1 (FIG. 1G) is an alternative binding domain that can replace MR1 in any of the above constructs to target EGFRvIII protein with higher affinity as described by Beers et al. The amino acid sequence changes made to create MR1-1 and the locations of these changes are indicated on FIG. 6.

The nucleotide and amino acid sequences for the antigen-binding region, MR1 scFv, of MR1 single chain antibody can be found in the GenBank database at Accession No. U76382 and AAB18787 respectively. The antigen binding region of MR1 binds specifically to EGFRvIII receptor. The MR1scFv useful for the construction of the chimeric proteins described herein is found at amino acids 4-240. Human CD8α is described in the SwissProt database at Accession No. P01732 and the nucleotide sequence of the human CD8α is found in Genbank Accession No. NM_{—}001768. The human CD8α hinge region is located at amino acids 135-182 and the transmembrane domain is located at amino acids 183-205 of the entire 235 amino acid polypeptide. Human CD28 is described in the SwissProt database at No. P10747; Genbank Accession No. J02988 and AAA60581. Regions of the human CD28 useful for the construction of the chimeric proteins described herein includes amino acids 113-220 of the full length 220 amino acid polypeptide, (this 113-220 amino acid region encompasses the extracellular region, TM and the intracellular signaling domain of human CD28), amino acids 180-220 (the intracellular signaling domain of human CD28), and amino acids 153-220 (the TM and the intracellular signaling domain of human CD28). The protein sequence of human OX-40 (TNFRSF4, CD134 antigen) is described in the SwissProt database at Accession No. P43489, and the nucleotide sequence is found in Genbank Accession No. X75962. The intracellular region including amino acids 242-277 of OX-40 can be used in the construction of chimeric proteins as described herein. The human zeta chain (CD3ζ) is described in the SwissProt database at Accession No. Q5VX14 and the nucleotide sequence is found in Genbank Accession No. AL359962. The intracellular region amino acids 52-163 of CD3Z can be used in the construction of chimeric proteins as described herein.

In one embodiment, the chimeric proteins include signal peptide sequences in the amino terminus of the protein to facilitate entry into the endoplasmic reticulum during co-translation. The signal peptide is MDWIWRILFLVGAATGAHSQVQ (SEQ. ID. No: 28). Other signal peptide sequences that can be used include MALPVTALLLPLALLLHAARP (SEQ. ID. No: 29), MLRLLLALNLFPSIQVTG (SEQ. ID. No: 30), MKWKALFTAAILQAQLPITEA (SEQ. ID. No: 31), and MCVGARRLGRGPCAALLLLGLGLSTVTG (SEQ. ID. No: 31).

Encompassed in the invention are nucleic acids encoding humanized chimeric proteins as disclosed herein (including SEQ. ID. Nos: 1, 3, 5, 7, 9, 11, 13, and 15). The nucleic acids comprise coding sequences for humanized chimeric proteins (SEQ. ID. Nos: 2, 4, 6, 8, 10, 12, 14, and 16). Also encompassed are nucleic acid constructs comprising the coding sequences for chimeric proteins described herein. The nucleic acid construct can be a vector carrying a nucleic acid encoding a humanized chimeric protein.

As used herein, the term “vector”, refers to a nucleic acid construct designed for delivery to a host cell or transfer between different host cells. As used herein, a vector may be viral or non-viral. The vector can also be a plasmid. The vector may be an expression vector for the purpose of expressing the encoded protein in the transfected cell. A viral vector can be any viral vector known in the art including but not limited to those derived from adenovirus, adeno-associated virus (AAV), retrovirus, and lentivirus. Recombinant viruses provide a versatile system for gene expression studies, gene transfer and genome integration, and therapeutic applications.

As used herein, the term “expression vector” refers to a vector that has the ability to incorporate and express heterologous or modified DNA fragments in a cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the coding sequence for a MR1-CD8-ζ and the various chimeric proteins described herein in place of non-essential viral genes. The vector and/or particle can be utilized for the purpose of transferring DNA, RNA or other nucleic acids into cells either in vitro or in vivo. Numerous forms of viral vectors are known in the art.

The expression vector should have the necessary 5′ upstream and 3′ downstream regulatory elements such as promoter sequences, ribosome recognition and binding TATA box, and 3′ UTR AAUAAA transcription termination sequence for the efficient gene transcription and translation in its respective host cell. The expression vector can have additional sequence such as 6X-histidine, V5, thioredoxin, glutathione-S-transferase, c-Myc, VSV-G, HSV, and FLAG tags which are incorporated into the expressed humanized chimeric proteins disclosed herein.

Examples of expression vectors are the strong CMV promoter-based pcDNA3.1 (Invitrogen) and pClneo vectors (Promega) for expression in mammalian cells; replication incompetent adenoviral vectors pAdeno X, pAd5F35, pLP-Adeno-X-CMV (Clontech), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells. The coding sequences of the chimeric proteins disclosed herein can be ligated into such expression vectors for the expression of the chimeric protein in mammalian cells.

“Nucleic acids” include DNA and RNA and chemical derivatives thereof, including molecules having a radioactive isotope or a chemical adduct such as a fluorophore, chromophore or biotin (“label”).

In particular embodiments, the nucleic acids described herein can be isolated. Isolated nucleic acids (e.g., DNAs and RNAs) can be purified from a natural source or can be made recombinantly. Nucleic acids referred to as “isolated” are nucleic acids purified to a state beyond that in which they exist in cells. They include nucleic acids obtained by suitable methods, and include substantially pure nucleic acids produced by chemical synthesis or by combinations of biological and chemical methods, and recombinant nucleic acids that have been isolated. The term “isolated” as used for nucleic acid molecules, indicates that the molecule in question exists in a physical milieu distinct from that in which it occurs as found in or as produced in a cell, but can have further cofactors or molecular stabilizers (for instance, buffers and/or salts) added. Such a nucleic acid can be part of a vector and/or such nucleic acid could be part of a composition, and still be isolated in that the vector or composition is not part of a cell.

An RNA equivalent of a DNA is a polymer of ribonucleotide units, and has the base U (uracil) at sites within a molecule where DNA has the base T (thymidine), but otherwise has the same nucleotide sequence as a strand of DNA, usually, the “plus” or coding strand of the DNA. The RNA equivalents of the DNAs described herein are aspects of the invention. Also included are the complementary minus strands of nucleic acids described as plus strands herein. Both single-stranded and double stranded nucleic acids are part of the invention.

The term “substantially pure” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. at levels of more than 90%, of more than 95% or of more than 99%) the component can be regarded as being in “pure form.” Biologically active substances of the present invention (including polypeptides, nucleic acid molecules, binding agents, moieties identified/identifiable via screening, etc.) can be provided in a form that is substantially free of one or more contaminants with which the substance might otherwise be associated. Thus, for example, they can be substantially free of one or more potentially contaminating polypeptides and/or nucleic acid molecules. They can be provided in a form that is substantially free of other cell components (e.g. of cell membranes, of cytoplasm, etc.). When a composition is substantially free of a given contaminant, the contaminant will be at a low level (e.g., at a level of less than 10%, less than 5%, or less than 1% on the dry weight/dry weight basis set out above). Substantially pure nucleic acids and vectors are part of the invention.

Generating Human T-Cells Expressing Humanized Chimeric Proteins

In one embodiment, the nucleic acid constructs or vectors carrying the coding sequence of the humanized chimeric proteins described herein can be introduced into human T-cells by various transfection methods that are known in the art. For example, the vectors can be electroporated into human T-cells or introduced into T cells using lipid-based transfection reagents such as lipofectamine (Invitrogen Inc.). Alternately, the vectors can be introduced into T-cells using the AMAXA based nuclefector by AMAXA Biosystems. Alternately, the vectors can coated on minute gold particles and “shot” into human T-cells using a gene gun (BioRad). Viral vectors can be also used.

In one embodiment, recombinant adenovirus can be used to transfect human T-cells and produce long-term expression of the chimeric protein. A simplified system for generating recombinant adenoviruses is presented by He T C. et. al. Proc. Natl. Acad. Sci. USA 95:2509-2514, 1998. The coding sequence of the chimeric protein disclosed herein is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In another embodiment, a recombinant lentivirus can be used for the delivery and expression of the chimeric proteins disclosed herein in mammalian cells. The HIV-1 based lentivirus can effectively transduce a broader host range than the Moloney Leukemia Virus (MoMLV)-base retroviral systems. Preparation of the recombinant lentivirus can be achieved using e.g. the pLenti4/V5-DEST™, pLenti6/V5-DEST™ or pLenti vectors together with ViraPower™ Lentiviral Expression systems from Invitrogen.

In yet another embodiment, the invention provides a recombinant adeno-associated virus (rAAV) vector for the expression of the chimeric proteins disclosed herein. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the chimeric DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

In one embodiment, a method for creating an expanded population enriched in genetically modified, humanized chimeric TCR protein expressing human T-cells which target an EGFRvIII protein is provided. Isolated human T-cells can be transfected with a vector carrying a nucleic acid construct that encodes the humanized chimeric EGFRvIII targeting T-cell receptor.

As used herein, the term “genetically modified” refers to the addition of extra genetic material in the form of DNA or RNA into the total genetic material in a cell. The terms “genetically modified T-cells” and “modified T-cells” are used interchangeably.

In another embodiment, the invention provides genetically modified, humanized chimeric TCR protein expressing human T-cells which target an EGFRvIII protein for the treatment of glioblastoma multiforme (GBM), breast, lung, ovarian, head and neck, or bladder tumors in a human subject.

In yet another embodiment, the invention provides genetically modified, humanized chimeric TCR protein expressing human T-cells which target an EGFRvIII protein for the killing of EGFRvIII-expressing cells found in glioblastoma multiforme (GBM), breast, lung, ovarian, head and neck, or bladder tumors in a human subject. Once the modified T-cells is attached to its target, it can release a variety of cytotoxic factors such as perforin, granulysin, and granzyme, a serine protease, that can enter target cells via the perforin-formed pore and induce apoptosis (cell death) by activation of cellular enzymes called caspases.

Accordingly, the invention provides a method of treating an EGFRvIII-expressing cancer in a human, comprising administering to a human diagnosed with an EGFRvIII-expressing cancer, a population of modified human T-cells as disclosed herein.

In one embodiment, the invention provides a population of modified human T cells for the treatment of cancer, the modified human T cells comprising a chimeric EGFRvIII targeting T cell receptor as disclosed herein. As used herein, the terms “chimeric EGFRvIII targeting T cell receptor”, “genetically modified, humanized chimeric TCR protein expressing T-cell”, “chimeric T-cell receptor” and “humanized chimeric protein” are used interchangeably.

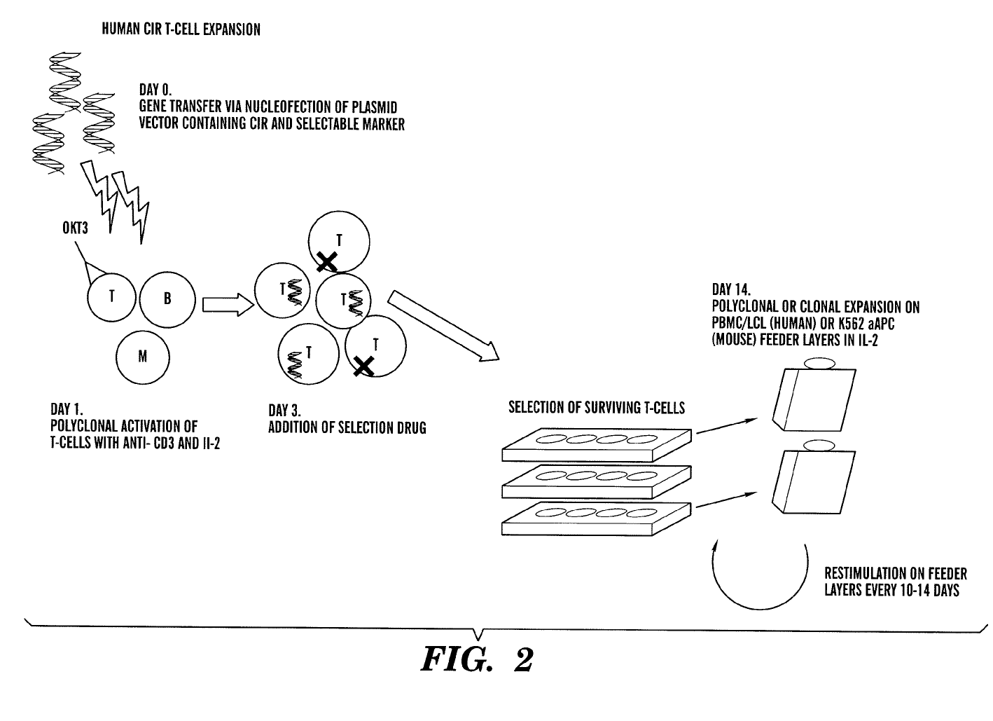

The population of modified human T cells are prepared from peripheral blood mononuclear cells (PBMCs) obtained from healthy human donors (allogenic donors) or from the patients diagnosed with glioblastoma multiforme (GBM), breast, lung, ovarian, prostate, head and neck, or bladder tumors (autologous donors). The PBMCs form a heterogeneous population of T-cells that can be CD4+, CD8+, or CD4+ and CD8+. The PBMC's also can include other cytotoxic lymphocytes such as natural killer (NK) cells. An expression vector carrying the coding sequence of a chimeric protein disclosed herein can be introduced into a population of human donor T cells or NK cells. Successfully transfected T-cells that carry the expression vector can be selected by drug resistance (eg. hygomycin resistance) and then further propagated to increase the number of these humanized chimeric TCR protein expressing T-cells (See FIG. 2) in addition to cell activation using anti-CD3 antibodies and IL-2 or any other methods known in the art. The standard procedure of trypsin and EDTA treatment that is well known in the art can be used to detach the expanded T-cells expressing the humanized chimeric TCR protein and harvest the T-cells for storage and/or preparation for use in a human subject. In a preferred embodiment, the in vitro transfection, culture and/or expansion of T cells be performed in the absence of non-human animal derived products such as fetal calf serum and fetal bovine serum. Since a heterogeneous population of PBMCs is transfected, the resultant transfected cells is a heterogeneous population of modified cells comprising a chimeric EGFRvIII targeting T cell receptor as disclosed herein. This population of modified cells are not a population of modified clonal cells and are also not derived from a mixture of modified clonal cells. As used herein, the term “clonal” refers to cells derived from a single cell. The single cell can be selected for propagation to give rise to many similar cells, all having originated from that one single selected first cell. The progeny of the single first cell are clonal cells. Thus, a population of cells can be a clonal population of cells deriving from the first single cell.

In one embodiment, a mixture of different expression vectors can be used in transfecting a human donor population of T-cells wherein each vector encodes a different chimeric protein as disclosed herein. The resultant transfected T-cells forms a mixed population of modified cells, with each modified cell expressing a different humanized chimeric TCR protein.

Accordingly, in one embodiment, the invention provides a method of treating an EGFRvIII-expressing cancer in a human, comprising removing T-cells from a human diagnosed with an EGFRvIII-expressing cancer, transfecting said T-cells with a vector comprising a nucleic acid encoding a chimeric T-cell receptor, thereby producing a population of modified human T-cells, and administering the population of modified T-cells to the same human. The EGFRvIII-expressing cancer is selected from a group consisting of glioma, breast cancer, lung cancer, prostate, head and neck, bladder and ovarian cancer.

In one embodiment, the invention provides a method of storing genetically modified, humanized chimeric TCR protein expressing human T-cells which target an EGFRvIII protein, comprising cryopreserving the T-cells such that the T-cells remain viable upon thawing. A fraction of the T-cells expressing the humanized chimeric TCR proteins can be cryopreserved by methods known in the art to provide a permanent source of such T-cells for the future treatment of patients afflicted with glioblastoma multiforme (GBM), breast, lung, ovarian, prostate, head and neck, and bladder tumors. When needed, the cryopreserved transfected T-cells can be thawed, grown and expanded for more such T-cells.

As used herein, “cryopreserving” refers to the preservation of cells by cooling to low sub-zero temperatures, such as (typically) 77 K or −196° C. (the boiling point of liquid nitrogen). Cryopreservation also refers to preserving cells at a temperature between 4-10° C. At these low temperatures, any biological activity, including the biochemical reactions that would lead to cell death, is effectively stopped. Cryoprotective agents are often used at sub-zero temperatures to prevent the cells being preserved from damage due to freezing at low temperatures or warming to room temperature.

Freezing is destructive to most living cells. Upon cooling, as the external medium freezes, cells equilibrate by losing water, thus increasing intracellular solute concentration. Below about 10°-15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotective agents which can be used include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), and polyethylene glycol (Sloviter, H. A. and Ravdin, R. G., 1962, Nature 196:548). The preferred cooling rate is 1° to 3° C./minute. After at least two hours, the T-cells have reached a temperature of −80° C. and can be placed directly into liquid nitrogen (−196° C.) for permanent storage such as in a long-term cryogenic storage vessel.

In one embodiment, the invention provides a pharmaceutical composition comprising a population enriched in genetically modified, humanized chimeric T-cell receptor (TCR) protein expressing human T-cells which targets an EGFRvIII protein and a pharmaceutically acceptable carrier.

As used herein, the term “pharmaceutical composition” refers to the active agent in combination with a pharmaceutically acceptable carrier of chemicals and compounds commonly used in the pharmaceutical industry. The term “pharmaceutically acceptable carrier” excludes tissue culture medium.

In another embodiment, a humanized chimeric TCR protein expressing T-cells can be prepared for treating a patient afflicted with cancer cells that expressed the mutant EGFRvIII, such as glioblastoma multiforme (GBM), breast or lung tumors, by infusing the T-cell preparation directly into the affected organ with tumors such as the brain, breast, and lungs. When the original PBMCs are derived from the recipient, the transfected T-cells are autologous to the recipient, and there is a much reduced chance of developing immune rejection of the genetically modified T-cells when such T-cells are reintroduced back into the patient.

Therapeutic Uses and Formulation

Cell surface receptors are attractive candidates for targeted therapy of cancer. Growth factors and their receptors play important roles in the regulation of cell division, development, and differentiation. Among those, the epidermal growth factor receptor (EGFR) was the first identified to be amplified and/or rearranged in malignant gliomas. The most common rearranged form, EGFR type III variant (EGFRvIII), has a deletion in its extracellular domain that results in the formation of a new, tumor-specific target found in glioblastoma multiforme (GBM), as well as in breast, ovarian, prostate, lung, head and neck squamous, and bladder carcinomas.

Common symptoms associated with GBM include but are not limited to progressive weakness, speech, visual loss, headaches, epileptic seizures, and hemorrhagic strokes. Once symptoms occur, the diagnosis of GBM is usually straight-forward. The tumor can be imaged by contrast-enhanced MRI scan. Progressive growth of the lesion on serial MR scans differentiates tumor from stroke. A PET scan showing increased uptake of glucose can also help separate a diagnosis of tumor from stroke. An open or needle biopsy provides tissue for microscopic diagnosis.

Common symptoms associated with lung cancer include but are not limited to cough, shortness of breath, wheezing chest pain hemoptysis, (bloody, coughed-up sputum), loss of appetite weight loss, and pneumonia (inflammation of the lungs). Other less common symptoms include generalized weakness, chills, swallowing difficulties, speech difficulties or changes (e.g., hoarseness), finger/nail abnormalities (e.g., “clubbing,” or overgrowth of the fingertip tissue), skin paleness or bluish discoloration, muscle contractions or atrophy (shrinkage), joint pain or swelling, facial swelling or paralysis, eyelid drooping, bone pain/tenderness, and breast development in men. During the physical examination, the physician may ask the patient to provide a sample of sputum for lung cells analyses and a sample of blood for lung cancer biomarkers analyses. Further diagnostic tests can include chest x-ray, chest MRI, bronchoscopy, CT/PET fusion imaging, needle biopsy, and bone scan.

The common symptoms for breast cancer include but are not limited to an abnormality on routine mammography, lumps—such as lumps in the armpit or above the collarbone that does not go away, breast discharge, nipple inversion, breast swellings, and changes in the skin overlying the breast. During the physical examination, the physician may request a mammography, an ultrasound, a MRI, and/or a biopsy of the breast.

The most common symptom of bladder cancer is blood in the urine (hematuria), which causes the urine to appear rusty or deep red in color. Other symptoms can include painful urination, frequent urination, having the urge to urinate, but without result. Bladder cancer can be diagnosed by cystoscopy, imaging or cytology procedures. Cystoscopy is the most common and reliable test for bladder cancer. A thin tube with a camera (cystoscope) is inserted into the bladder through the urethra to provide a view of the suspicious area. The cystoscope can also be used to take a tissue sample for biopsy, and to treat superficial tumors without the need for surgery.

Prostate cancer strike one in every six men in America. The common symptoms include a need to urinate frequently, especially at night, difficulty starting urination or holding back urine, weak or interrupted flow of urine, painful or burning urination, difficulty in having an erection, painful ejaculation, blood in urine or semen, or frequent pain or stiffness in the lower back, hips, or upper thighs. Blood DRE and PSA level changes can also indicate prostate cancer. A biopsy where a needle is inserted into the prostate to take small samples of tissue, often under the guidance of ultrasound imaging, is often performed in order to make a definitive diagnosis of the disease.

Diagnosis of ovarian cancer is often difficult mainly because the symptoms: abdominal pain or swelling, gastrointestinal symptoms (gas, constipation, diarrhea, and others), or pelvic pain are often attributed to other ailments. These symptoms appear to be more digestive problems and are not pointing to the ovaries, hence ovarian cancer is often diagnosed when the cancer is at a later stage than other cancer types. When abdominal symptoms persist, a physician should consider and perform an ultrasonic evaluation of the ovaries in a female patient to rule out ovarian cancer. Laparoscopy surgery to obtain a biopsy sample for cancer staging can be performed when cancer is suspected.

Most head and neck cancers are squamous cell carcinomas, originating from the mucosal lining (epithelium) of the upper aerodigestive tract, including the lip, oral cavity (mouth), nasal cavity, paranasal sinuses, pharynx, and larynx. Head and neck cancers often spread to the lymph nodes of the neck, and this is often the first (and sometimes only) manifestation of the disease at the time of diagnosis. Symptoms include enlarged lymph nodes on the outside of the neck, a sore throat or a hoarse sounding voice, difficult or painful swallowing, difficulty speaking, persistent earache, some numbness or paralysis of the face muscles, neck pain, weight loss, bleeding in the mouth, and sinus congestion. A patient usually presents to the physician complaining of one or more of the above symptoms The patient will typically undergo a needle biopsy of this lesion, and the sample is sent for histopathologal analysis.

As used herein, a tumor is an abnormal growth or mass of tissue. Often tumors are caused by mutations in DNA of cells, which interfere with a cell's ability to regulate and limit cell division. When the ability to regulate and limit cell division is lost or diminished, cell division progresses unchecked, resulting in a localized abnormal mass of cells, a tumor.

Tissues obtained from the biopsies can be analysed for EGFR and EGFRvIII expression by immunohistochemistry as described in Heimberger A B. et. al., 2005. Briefly, the primary antibody for EGFR detection was the monoclonal mouse anti-human pan-EGFR clone 528 (Oncogene Research, Product, San Diego, Calif.; 1:50 dilution; ref. 19) and for EGFRvIII detection was a rabbit anti-human polyclonal antibody (Zymed, San Francisco, Calif.; 1:1,200 dilution). For EGFRvIII staining, microwave antigen retrieval was done by placing the slides of the tissue in 50 mmol/L citrate buffer (pH 6.0) and microwaving for 12 minutes at full power and 10 minutes at 20% power followed by cooling for 15 minutes and two to three 5-minute washes in PBS. For EGFR staining, pretreatment consisted of placing 0.025% trypsin on the tissue and incubating for 30 minutes at room temperature. Primary antibodies, diluted in PBS/10% serum, were applied to the sections in a humid chamber overnight at 4° C. Tissue sections were washed two to three times in PBS, and secondary antibodies were applied using the Dako Envision kit (Carpinteria, Calif.), according to the manufacturer's instructions. Detection of bound secondary antibody was done with diaminobenzadine for 5 minutes. Sections were then counterstained with hematoxylin and mounted. As a control tissue, healthy tissue from surround the excised tumor is also obtained for analyses. Other diagnostic methods known in the clinical art include reverse transcription PCR, real-time PCR and western blot analysis.

The EGFRvIII does not naturally occur in healthy human tissues. When there is a positive detection of EGFRvIII in a tumor biopsy tissue over the background detection of EGFRvIII in a healthy human tissue, the tumor is diagnosed as expressing EGFRvIII. The positive detection of EGFRvIII in a tumor tissue is at least 5% greater than the background detection of EGFRvIII in a healthy human tissue

Once the presence of EGFRvIII expressing tumor cells has been determined, a genetically modified, humanized chimeric TCR protein expressing human T-cells which target an EGFRvIII protein can be administered to the subject to treat the GBM, breast or lung tumor. The treatment of GBM, breast or lung tumor in a subject comprises killing the EGFRvIII-expressing cells in the subject with the genetically modified, humanized chimeric TCR protein expressing human T-cells which targets an EGFRvIII protein. The killing of EGFRvIII-expressing tumor cells means that the number of viable tumor cells is decreased relative to the untreated condition. There can be a reduction in the size of the tumor. The inhibition of tumor growth can be observed, for example, by radiologic methods and/or imaging methods, and can include a reduction in growth rate and/or a reduction in size and/or number of tumors (versus untreated condition).

In one embodiment, the invention provides a method of killing EGFRvIII-expressing cells in a human comprising administering to a subject a T cell comprising a vector comprising a nucleic acid encoding a humanized chimeric TCR protein that targets an EGFRvIII protein. In one embodiment, the EGFRvIII-expressing cells in a human comprise GBM cells, breast, ovary, prostate, neck and neck squamous carcinoma, bladder and lung cancer cells.

In one embodiment, the invention provides a method of treating GBM in a subject in need thereof, comprising administering a genetically modified, humanized chimeric TCR protein expressing human T-cell which targets an EGFRvIII protein. Successful treatment can be measured in terms of lessening the symptoms associated with GBM as described supra, a reduction in the size of the glioblastoma, at least 5% reduction in the size of the tumor mass, or if there is zero growth of tumor mass, non-reoccurrence of new glioblastoma after removal of the primary tumor and/or prolonging the survival time after positive diagnosis beyond the average of 15 months.

In one embodiment, the invention provides a method of treating lung cancer in a subject in need thereof, comprising administering a genetically modified, humanized chimeric TCR protein expressing human T-cell which targets an EGFRvIII protein. Successful treatment can be measured in terms of lessening the symptoms associated with lung cancer as described supra, a reduction in the size of the lung tumor, at least 5% reduction in the size of the tumor mass, or if there is zero growth of tumor mass, non-reoccurrence of new tumors after removal of the primary tumor, and/or prolonging the survival time after positive diagnosis.

In one embodiment, the invention provides a method of treating breast cancer in a subject in need of, comprising administering a genetically modified, humanized chimeric TCR protein expressing human T-cell which targets an EGFRvIII protein. Successful treatment can be measured in terms of lessening the symptoms associated with breast cancer as described supra, a reduction in the size of the breast tumor, at least 5% reduction in the size of the tumor mass, or if there is zero growth of tumor mass, non-reoccurrence of new tumors after removal of the primary tumor, and/or prolonging the survival time after positive diagnosis.

Lactated Ringer's solution is a solution that is isotonic with blood and intended for intravenous administration. The pharmaceutical formulation for administering a genetically modified, humanized chimeric TCR protein expressing human T-cell is preferably a sterile saline or lactated Ringer's solution.

In one embodiment, genetically modified, humanized chimeric TCR protein expressing human T-cells which target an EGFRvIII protein can be administered to a human in need thereof by any suitable route, and means, for example, intravenous, intra-arterial intracranial, intracerebrospinal, intratumoral, peritoneal, by injection, by catheter, by implantation with or without a matrix or gel material, or by gradual delivery device. In one embodiment, the T-cells as described herein can be administered directly by injection. If the solid tumors are accessible by injection, genetically modified T-cells can be administered by injection directly to the tumor mass as a pharmaceutical formulation. The preferred formulation is sterile saline or Lactated Ringer's solution.

In one embodiment, T-cells as disclosed herein can be administered alone or in combination with other pharmaceuticals, polypeptides and/or cells. For example, T-cells can be administered in combination with a VEGF-CIR expressing T-cell, or other anti-VEGF therapies such as soluble VEGFR.

Compositions comprising T-cells of the invention can be administered parenterally. Such compositions can include aqueous sterile injectable suspensions of cells. These can contain antioxidants, buffers, antibiotics and solutes that render the compositions substantially isotonic with the blood of an intended recipient. Other components that can be present in such compositions include water, polyols, glycerine and vegetable oils, and nutrients for cells, for example. Compositions adapted for parenteral administration can be presented in unit-dose or multi-dose containers, in a pharmaceutically acceptable dosage form. Such dosage forms, along with methods for their preparation, are known in the pharmaceutical and cosmetic art. HARRY'S COSMETIC LOGY (Chemical Publishing, 7th ed. 1982); REMINGTON'S PHARMACEUTICAL SCIENCES (Mack Publishing Co., 18th ed. 1990).

In one embodiment, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In one embodiment, other ingredients can be added to pharmaceutical formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The route of administration, dosage form, and the effective amount of T-cells to be administered may vary according to the expression level of the chimeric TCR proteins and the potency of the cell killing by the T-cell and according to the treatment location. The selection of proper dosage is well within the skill of an ordinarily skilled physician.

This invention is further illustrated by the following examples which should not be construed as limiting. The contents of all references cited throughout this application, as well as the figures and table are incorporated herein by reference.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

### EXAMPLES

### Materials and Methods

Target cell lines—The wild-type human glioma cell line U87MG was obtained from American Type Culture Collection (Rockville, Md.) the Epidermal Growth Factor Receptor (EGFR) vIII variant glioma cell line, termed U87EGFRvIII, was a kind gift of Dr. Xandra Breakfield (Massachusetts General Hospital, Boston, Mass.). Firefly Luciferase expressing EGFRvIII positive U87 glioma cell line (U87vIII-FFlucZeo) was created by infecting the original U87vIII parental cell line with replication-defective lentivirus encoding Firefly Luciferase-Zeocin using a five-plasmid transfection procedure as described previously (G. J. Murphy et al. Nature Medicine 2006 August).

Generation of human effector T-cell clones. Day 0: Peripheral blood mononuclear cells (PBMCs) were obtained from healthy donors), prepared via Ficoll gradient separation and washed in phosphate buffered saline containing ethylenediamine tetracetic acid (PBS-EDTA). Total mononuclear cells were plated in T-cell growth media (TCGM) (10⁶ cells/ml) in 75 cm² flasks in the presence of OKT3 (a monoclonal antibody that binds to the CD3 complex on T-cells) and rhIL-2 (recombinant human interleukin 2) at 50 U/ml. Day 3: T-cells were electroporated in an Amaxa nucleofector device using 5 μg/linearized CIR expression plasmid DNA per 1×10⁶ cells. Days 3-14: After electroporation, cells were resuspended in TCGM and 25 U/ml rhIL-2 and on day 5 placed under selection with 0.2 mg/ml hygromycin. Every other day, cells were supplemented with IL-2 (25 U/ml) and 25 ml of cell free medium was replaced with fresh medium. Days 14-28: on day 14, cloning was initiated. Electroporated bulk mononuclear cells were passed through a Ficoll gradient and viability assessed. Cells were combined with 5×10⁶/ml irradiated (3500 cGy; cGy=centiGreys) PBMC feeder cells and 1×10⁶/ml irradiated (8000 cGy) TM-LCL feeder cells with rhIL-2 and OKT3 and disbursed into 96 well plates for cloning by limiting dilution. Day 28: wells were scored for positive clones using half of each microculture for cytotoxicity testing and flow cytometry. Clones demonstrating cytotoxicity and a CD4 negative profile were further expanded by transferring the remaining half of each positive well into 2 ml cultures (24-well plates) containing rhIL-2 and OKT3. After further growth into multiple 2 ml cultures, the cells were transferred into 25 cm² flasks for further expansion in rhIL-2 and OKT3. After 4-5 14 day cycles of expansion in rhIL-2 and OKT3 on irradiated PBMC and LCL (lymphoblastoid cell line) feeders, cells were frozen for future use or used directly in adoptive transfer experiments.

Detection of Gene Expression by RT-PCR-Gene expression was tested by RT-PCR (reverse transcription polymerase chain reaction), using primers to amplify a 496 by fragment of the chimeric construct. Total RNA was extracted from PMG-MR1-CD8-ζ (M8Z), PMG-MR1-B-CD8-ζ (MR1-B) and PMG-MR1-CD8-Delζ (MR1-DelZ) nucleofected and non-nucleofected control human PBMCs on day 10 of stimulation cycles 2-10, using Absolutely RNA Miniprep Kit (Stratagene, LaJolla Calif.) according to the manufacturer's protocol. RT-PCR was performed using Reverse-IT One Step Kit (ABgene, Rochester N.Y.). Each reaction contained 1.2 μg of total RNA as a template and the following primers in 10 μM concentration: Sense M8Z: 5′-CGT GCC TCT TAC ATT CGG TGA T-3′ (SEQ ID NO:18); anti-sense M8Z: 5′-CCT CCG CCA TCT TAT CTT TCT G-3′ (SEQ ID NO:19) for detecting MR1-CD8-t; sense MR1-B: 5′-CTC TCC TG GTA ACC-3′ (SEQ ID NO:20); antisense MR1-B: 5′-CCT CCG CCA TCT TAT CTT TCT G-3′ (SEQ ID NO:21); sense MR1-DelZ: 5′-CGT GCC TCT TAC ATT CGG TGA T-3′ (SEQ ID NO:22); antisense MR1-DelZ #1: 5′-TAT CGC TCA GCG CGC GGG AGG CTC TGC GCT-3′ (SEQ ID NO:23); antisense MR1-DelZ #2: 5′-CCT CCG CCA TCT TAT CTT TCT G-3′ (SEQ ID NO:24) (same as antisense for M8Z); sense β-Actin ns: 5′-TGA CGG GGT CAC CCA CAC TGT GCC CAT CT-3 (SEQ ID NO:25); anti-sense β-Actin: 5′-CGA AGC ATT GCG GTG GAC GAT GGA GC-3 (SEQ ID NO:26) to amplify β-Actin as a positive control for RNA isolation. To eliminate residual contaminating DNA, 1 U of RNase free DNase I (Stratagene, LaJolla Calif.) was added to each sample during the RNA purification procedure.

Flow Cytometry—Surface expression of the chimeric cassettes was analyzed by flow cytometry using PE (phycoerythrin) conjugated monoclonal antibody against the c-myc epitope, located 3′ of the MR1 scFv region and 5′ of the CD8α hinge region. Distribution of CD4+/CD8+ T-cells within freshly isolated PBMC and culture chimeric populations was determined by FACS analysis using allophycocyanin (APC) conjugated anti-human CD4 and FITC-conjugated (FITC=fluorescein isothiocyanate) anti-human CD8 monoclonal antibodies.

Cytotoxicity assays—Cytotoxicity was studied using europium release assays (von Zons et al., 1997). Briefly, target tumor cells were incubated for 5-10 min with Delphia® BATDA labeling reagent (PerkinElmer, Wellesley Mass.), which is hydrolyzed to TDA upon penetrating the cell membrane. A two hour co-incubation of targets, irrelevant controls, and CIR expressing effectors at varying ratios was performed, at which time supernatants were harvested, mixed with 200 μl europium solution, which forms a stable complex with released TDA, and read in a Wallac Victor 3 luminometer (PerkinElmer) using time resolved fluorometric detection. Results were expressed in specific cytotoxicity [100*(experimental release-spontaneous release)/(total release-spontaneous release)].

### Example 1

### Human CD8+ T-Cells can be Engineered to Express a Chimeric Receptor

Chimeric T-cell receptors are schematically represented in FIGS. 1A-1G. MR1-CIR includes the scFv MR1 which binds EGFRvIII, the CD8alpha hinge and transmembrane domain, and the zeta chain of the T-cell receptor. MRB-CIR refers to a “binding” mutant in which the extracellular domain of the MR1scFv is altered and does not bind EGFRvIII, yet has an intact signaling domain. This provides a control that has signaling capacity but no binding capacity. MR1-delZ-CIR provides intact binding, but a stop codon is placed prior to any of the signaling immunoreceptor tyrosine-based activation motifs (ITAMs) in the TCR-zeta. This provides a control CIR which can bind to target cells but does not signal.

All CIR expression cassettes were cloned into the plasmid IL-13 CIR/HyTK/ff(luc), provided by Dr. M. Jensen, replacing the IL-13 moiety with EGFR targeting moieties. This plasmid contains cytomegalovirus (CMV) driven CIR expression coupled with internal ribosome entry site (IRES) driven expression of the HyTK fusion gene (to allow for hygromycin selection of stably transfected T-cell clones and if necessary, due to toxicity, ganciclovir deletion of adoptively transferred cells).

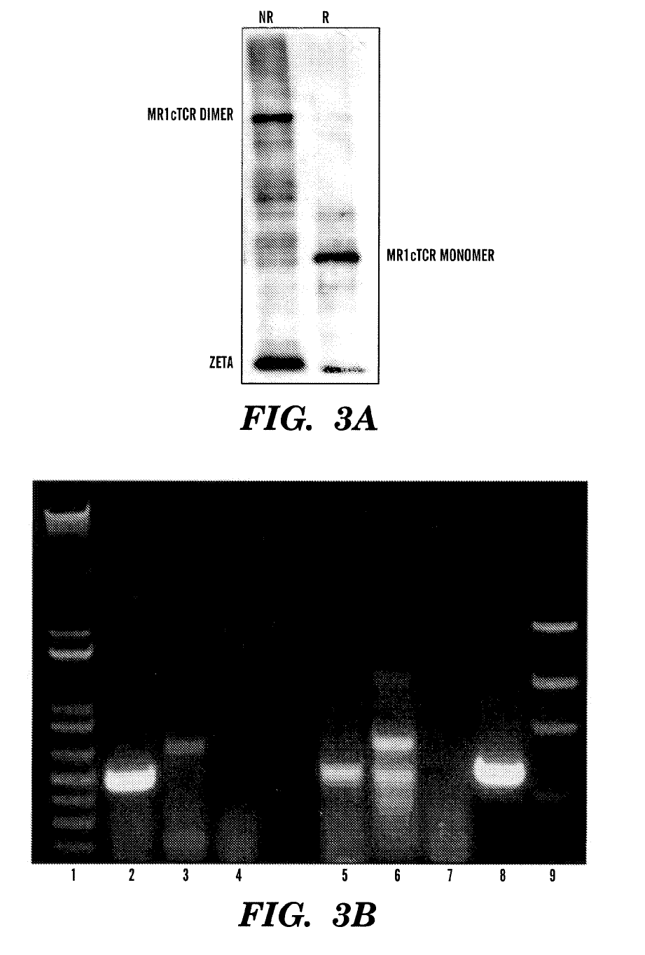

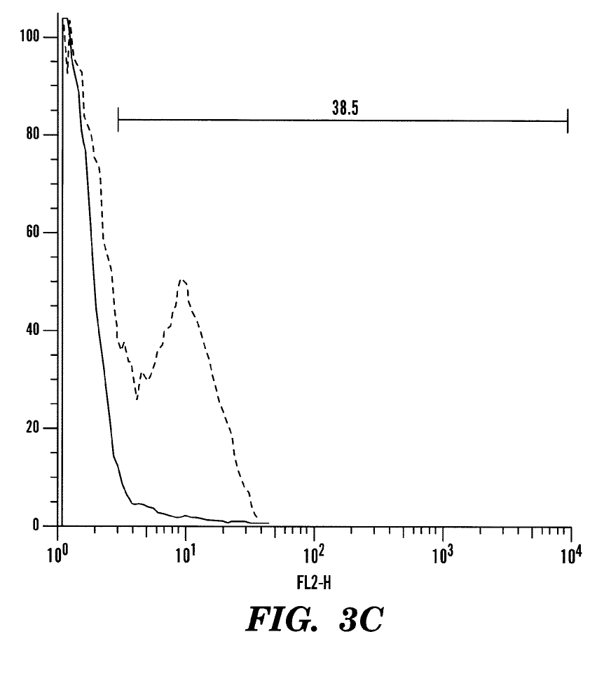

Using a system (FIG. 2) developed by Dr. Jensen which allows for plasmid based transduction, selection and culturing of CIR+ CD8+ cells, human PBMC's were transfected with the MR1, MRB, and MR-delZ CIRs and expanded them over several biweekly cycles (up to 10 cycles). Each cycle includes restimulation on LCL and peripheral blood mononuclear cells (PBMC) feeder layers in the presence of IL-2 and hygromycin selection. The cells expanded significantly and continued to express the receptor after several stimulation cycles as assessed by western blot, RT-PCR and fluorescence activated cell sorting (FACS) analysis (FIGS. 3A-3C).

### Example 2

### Human T-Cells Expressing MR1-CD8zeta-CIR Lyse Cells Expressing EGFRvIII

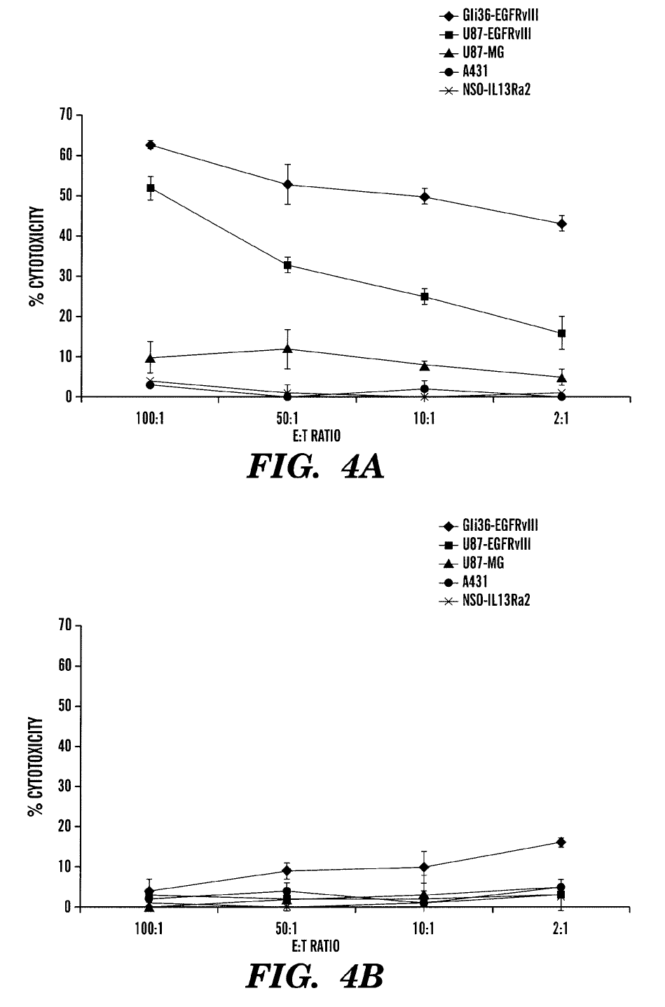

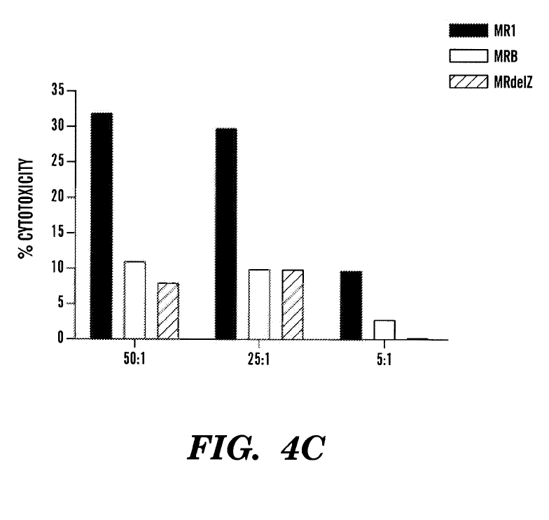

Significant cytotoxicity was noted against cells that express EGFRvIII, but not against other non-EGFRvIII expressing cells. In addition, full cytotoxic activity required both the presence of the binding epitope (the MR1 scFv) and the signaling domain (TCR-zeta) as the MRB and MR1-delZ variations did not show cytotoxic activity. See FIGS. 4A-4C.

### Example 3

### Human T-Cells Expressing the MR1-CD8-ζ CIR Lyse Human Glioma Cells Expressing EGFRvIII

In an experiment to test for lysis in cultured cells, 50,000 U87 or U87EGFRvIII tumor cells were mixed with 500,000 human T-cells expressing either EGFRvIII directed CIR (MIR1-CD8-ζ) or a control CIR (MRB-CIR) which does not bind EGFRvIII. After 72 hours, complete lysis of the human glioma cells expressing EGFRvIII by the human T-cells expressing MR1-CD-ζ CIR was seen by light microscopy, but not in control cultures.

### Example 4

### Human CD8+ MR1-CIR T-Cells Specifically Secrete INF-γ Upon Engagement of Target Cells that Express EGFRvII

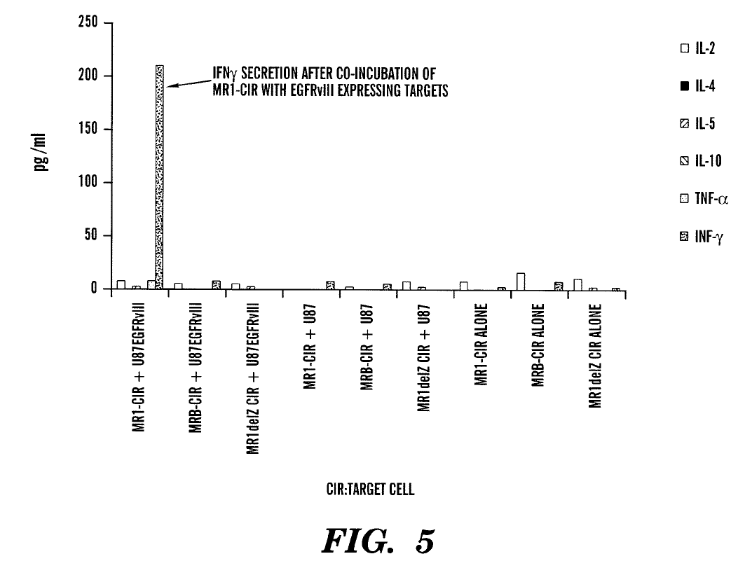

Human Th1/Th2 cytokine bead array analysis was performed. Human T-cells were transfected with CIRs encoding the full length MR1-CIR, a binding mutant MRB-CIR, or a signaling mutant MR1delZ CIR, and expanded over 10 weeks in culture. Expression was confirmed by RT-PCR and FACS in all samples. T-cells were then co-incubated with wild-type U87 glioma target cells, U87-EGFRvIII cells, or alone and assayed for IL-2, IL-4, IL-5, IL-10, TNFα, and IFN-γ expression. As seen in FIG. 5, there was marked and specific expression of IFN-γ in response to only the EGFvIII-expressing target cells, confirming the specificity of interaction with EGFvIII+ targets.

### Example 5

### Use of an Artificial Antigen Presenting Cell (APC) Expressing 41BBL and Preloaded with Anti-CD3 and Anti-CD28 Antibodies Permits Long-Term Expansion of Murine CD8+ T-Cells

The retroviral transduction system, with a prestimulation phase with IL-2, anti-CD3, and anti-CD28 to induce cell division prior to transduction of bulk populations, led to short duration of gene expression and rapid loss of cell populations after in vivo adoptive transfer with a duration of expression of less than two weeks as assessed by in vivo bioluminescence.

An artificial APC system described by Yan et al (2004) was used for long term culture and expansion of murine CD8+ cells. This involves culturing freshly harvested murine splenocytes on artificial APCs that express 4-1BBL and are precoated with anti-CD3 and anti-CD28 antibodies. The population of both non-transfected (>350 million) and transfected cells (>100 million cells) was expanded over several weeks. These cells express the CIR, as seen by RT-PCR, even after several weeks in culture.

### Example 6

### Human CD8+ T-Cells Co-Expressing a Chimeric Receptor and Luciferase can be Tracked Non-Invasively by Bioluminescence and Histologically to the Site of Intracranial Tumors

Mice were implanted with 50,000 U87 IL13R2a expressing tumor cells intracranially. Twenty-one days later, mice were treated with IV (tail vein) injection of 2E6 IL-13 CIR cells with co-expression of firefly luciferase (gift of Dr. M. Jensen). Sixteen days later (post implantation day 38), mice were imaged. Two of three treated animals showed excellent co-localization of T-cell derived luciferase signal in the region of the tumors. Immunostaining with fLuc antibody revealed T-cells localizing to the viable tumor periphery within the brain.

### Example 7

### Human CD8+ T-Cells Carrying Humanized MR1-CD8-ζ Restricted Intracranial Tumor Growth in Mice

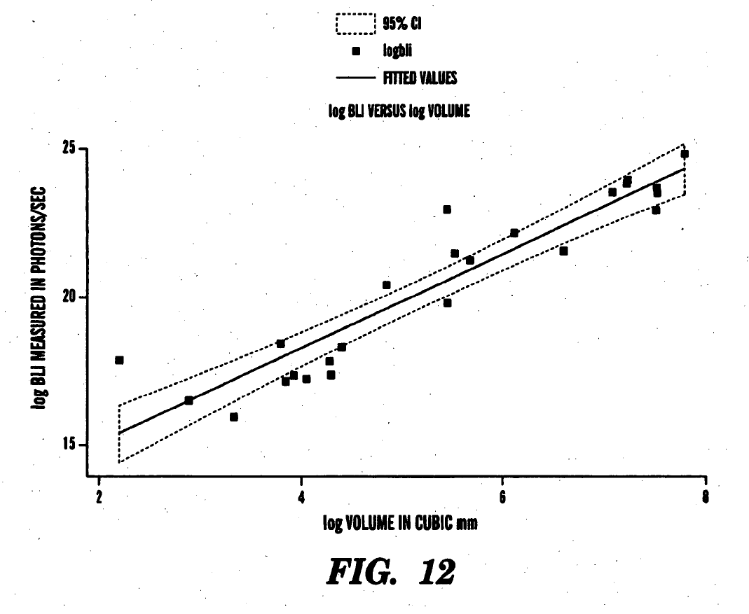

Four mice were injected intracranially with either 1×10⁵ or 5×10⁴ U87 firefly luciferase expressing tumor cells and the growth of the human glioblastoma xenograft was monitored weekly for four weeks for the increase in tumor size (data not shown). The measurements were in bioluminence imaging (BLI) and by magnetic resonance imaging (MRI). The BLI value correlated well with the observed MRI results; there was increased BLI with increasing tumor size as observed by MRI (FIG. 12).

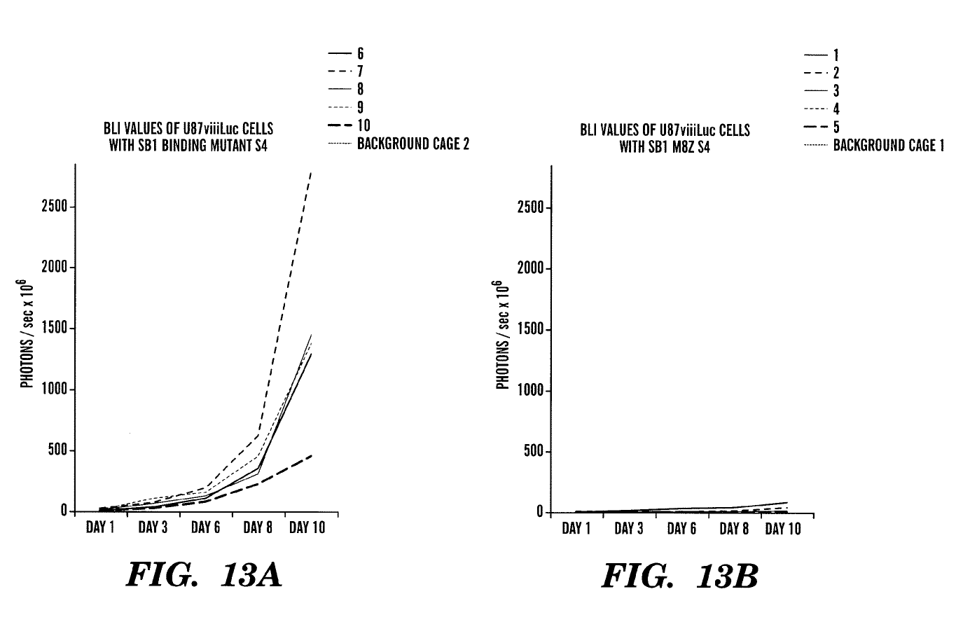

Five mice were injected intracranially with 5×10⁴ U87 tumor cells bearing the mutant EGFRvIII and expressing the firefly luciferase gene (U87vIIILuc) and the growth of the human glioblastoma U87vIIILuc xenograft was monitored daily for 10 days for an increase in tumor size as measured bioluminence imaging (BLI) (FIG. 13). These mice were also injected (intracerebrally) with humanized MR1-CD8-ζ T-cells (for mouse # 1-5) or humanized MR1-B-CD8-ζ T-cells (for mouse # 6-10). The deletion in the antigen binding region of MR1-B-CD8-ζ prevents the chimeric protein from binding the EGFRvIII on the tumor cells. The growth and expansion of the U87vIIILuc tumor cells were greatly restricted in the presence of the humanized MR1-CD8-ζ T-cells (for mouse # 1-5) (FIG. 13B) but not in the presence of the humanized MR1-B-CD8-ζ T-cells (for mouse # 6-10) (FIG. 13A). The U87vIIILuc tumor cells started to grow exponentially between day 8-10 after implantation of these tumor cells.

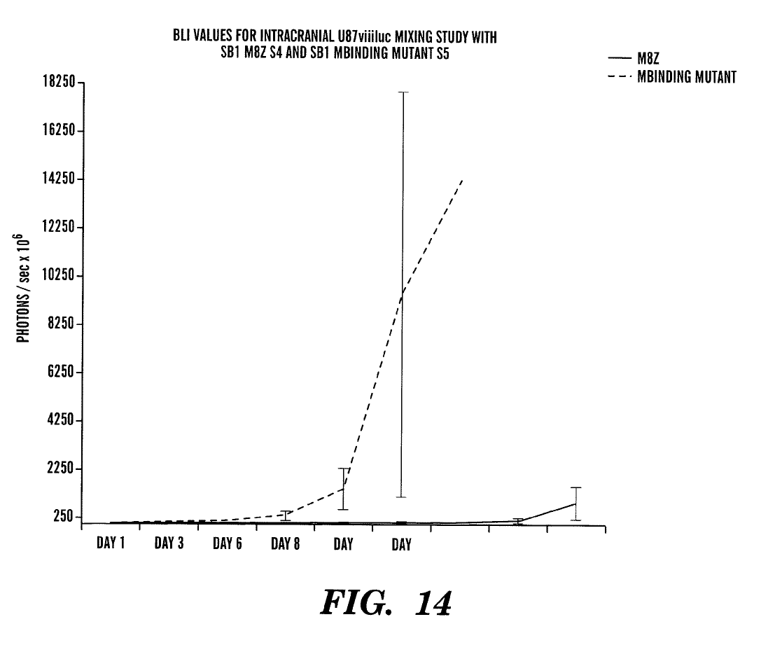

When U87vIIILuc tumor cells are mixed with humanized MR1-CD8-ζ T-cells immediately before the mixture of tumor cell and T-cells was implanted into mice, the same restriction on tumor cell growth and expansion was observed in these intracranially transplanted mice (FIG. 14). However, in a similar experiment where humanized MR1-B-CD8-ζ T-cells were used, there was no restriction on tumor cell growth, and exponential growth was observed between day 8-10 after implant of the tumor cell-ζ T-cell mixture. The presence of humanized MR1-CD8-ζ T-cells was able to limited the growth of the EGFRvIII expressing tumor cells in the mice.

### Example 8

### Human CD8+ T-Cells Carrying Humanized MR1-CD8-ζ Prolong Survival of NOD-SCID Mice with Intracranial Tumor Implants of U87vIIILuc Tumor Cells

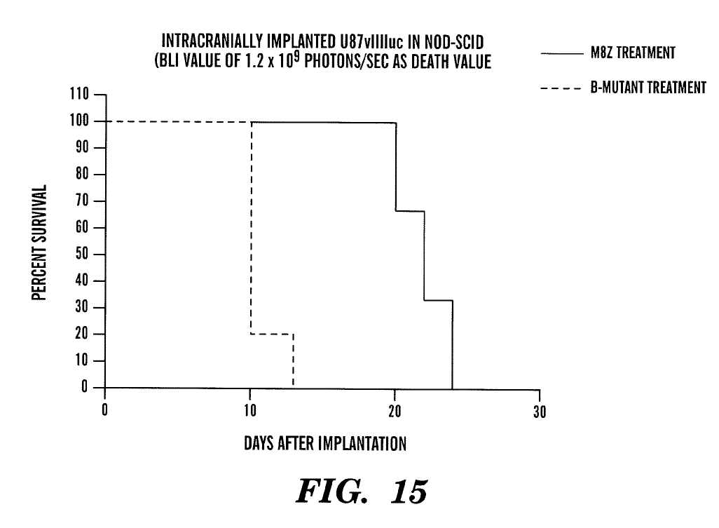

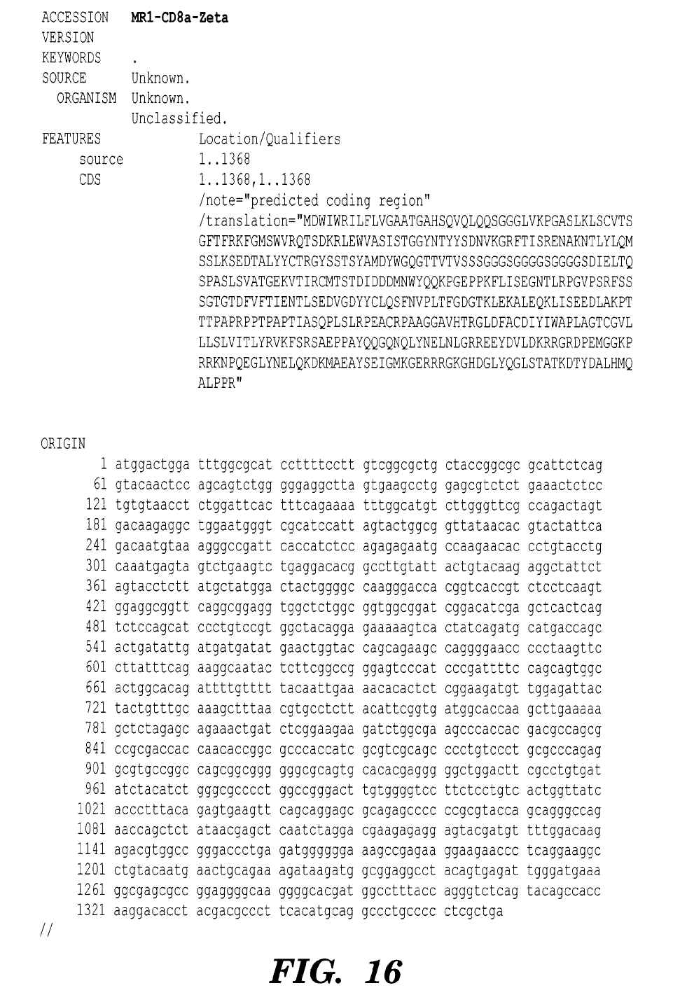

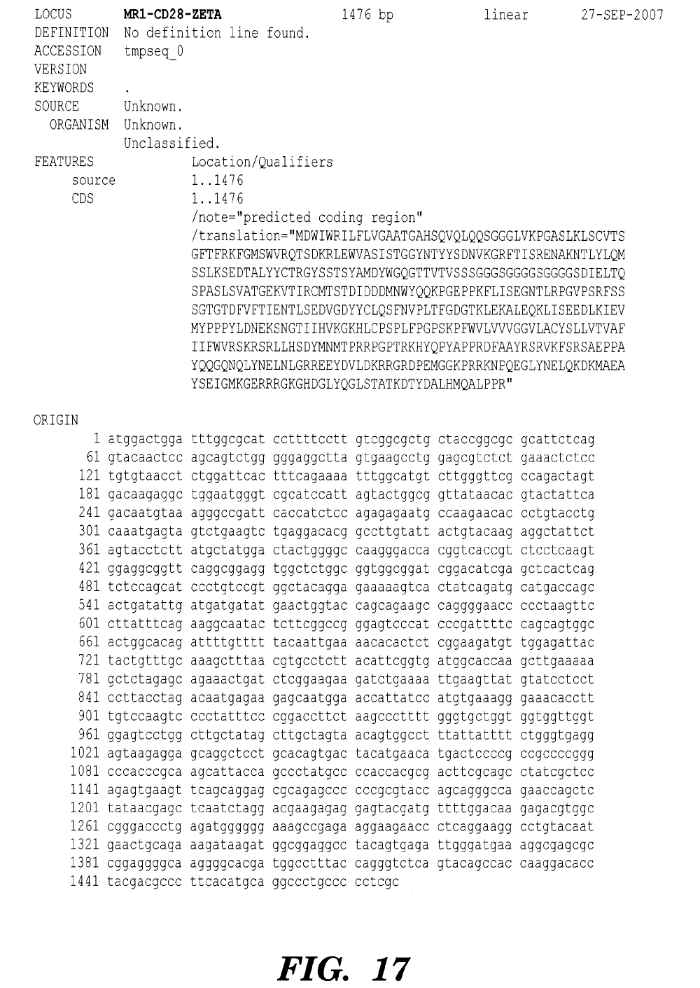

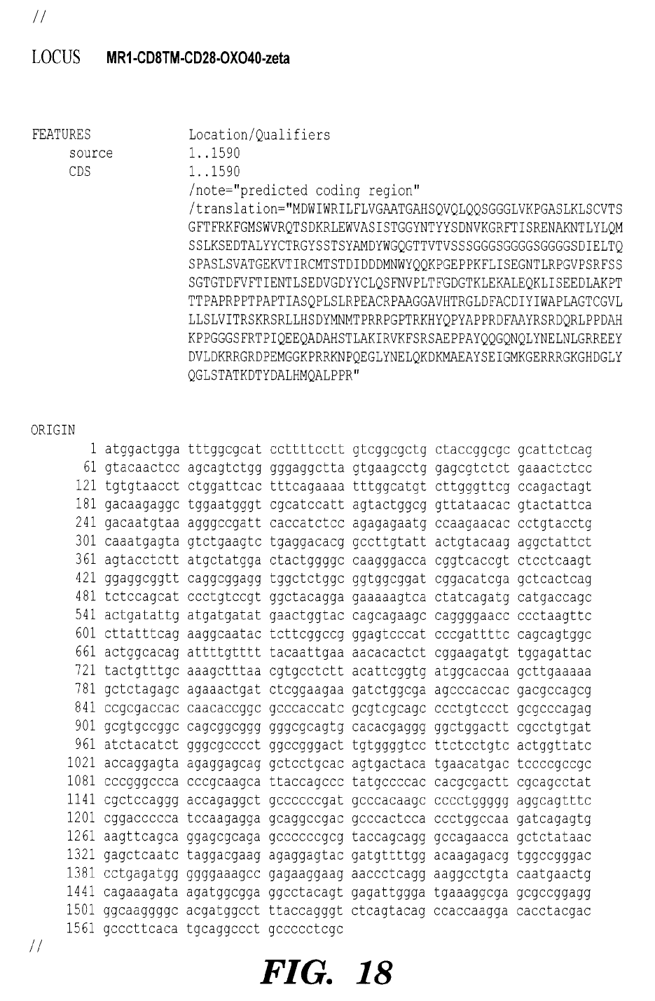

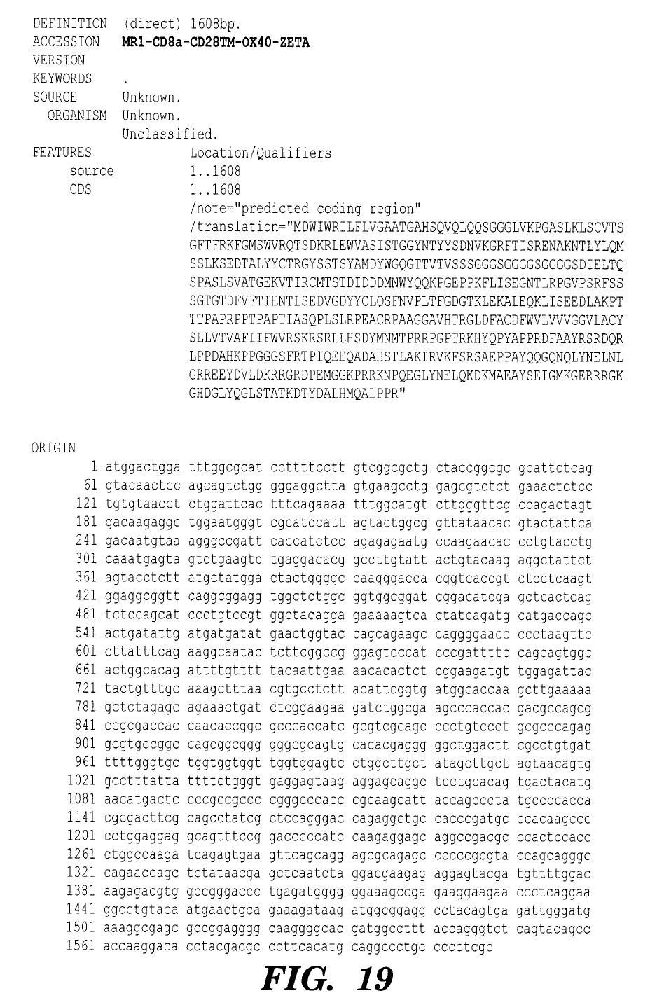

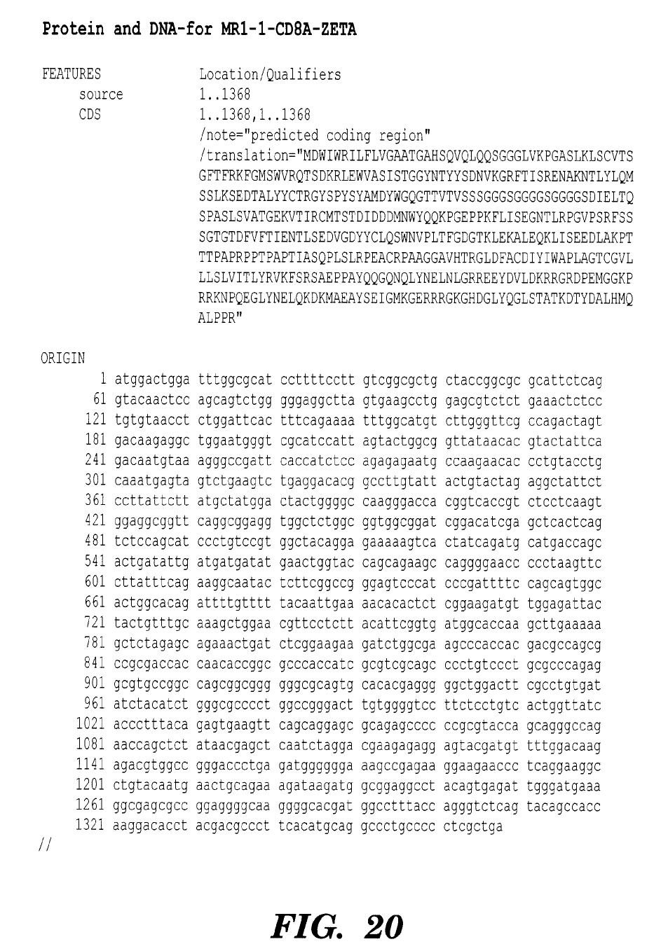

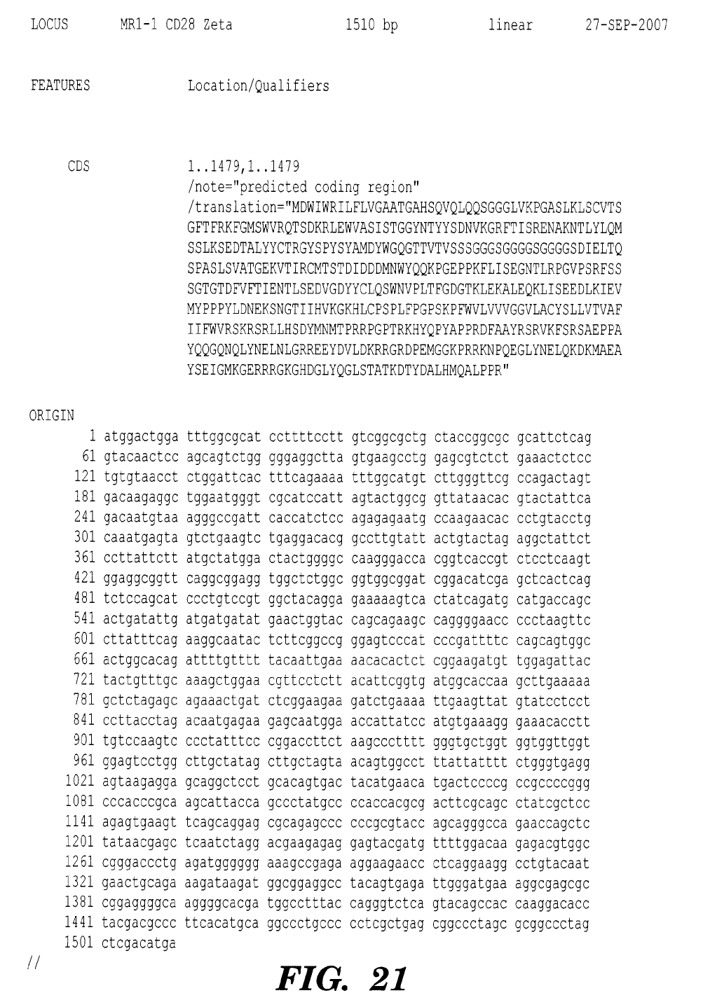

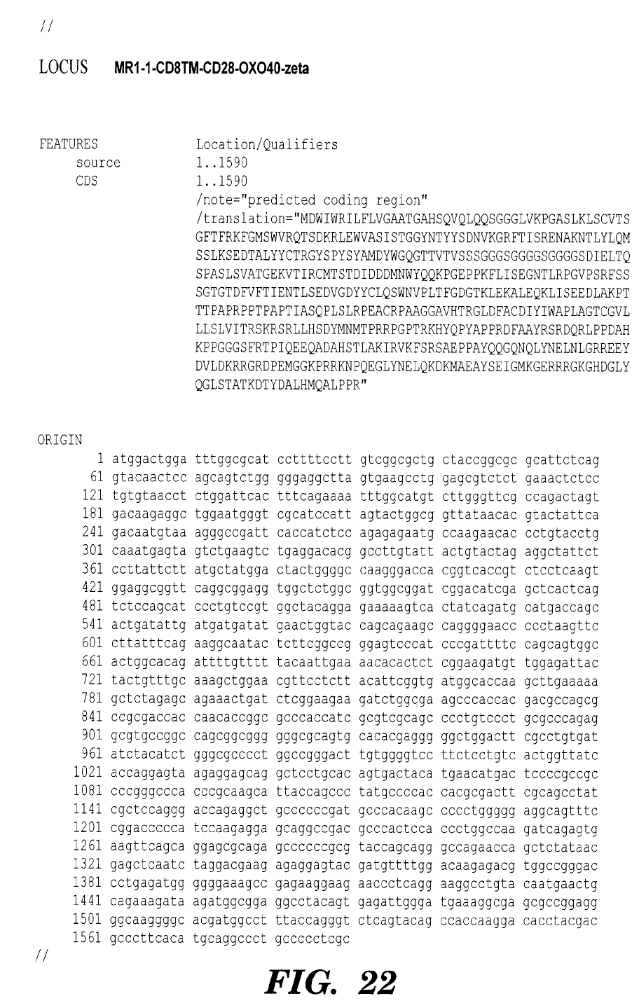

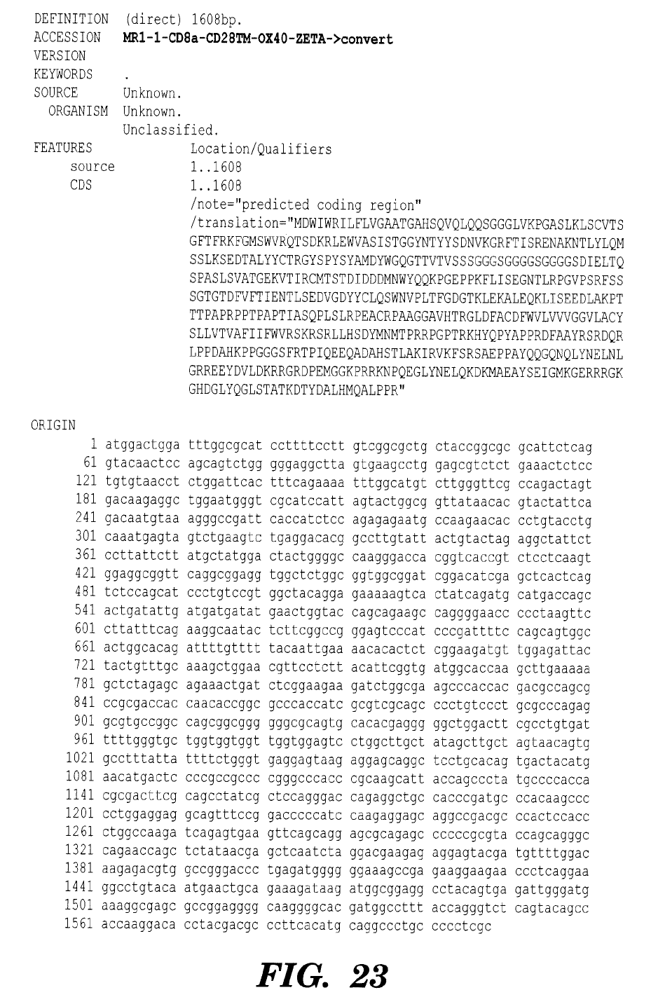

Mice were injected intracranially with 5×10⁴ U87 bearing the mutant EGFRvIII and expressing the firefly luciferase gene tumor cells (U87vIIILuc) and the health of the implanted mice was monitored daily for 30 days as scored by percentage survival (FIG. 15). These mice were also injected (intracerebrally) with humanized MR1-CD8-ζ T-cells or humanized MR1-B-CD8-ζ T-cells. Mice receiving the humanized MR1-CD8-ζ T-cells survived on average twice as long as the mice receiving the humanized MR1-B-CD8-ζ T-cells. The presence of humanized MR1-CD8-ζ T-cells was able to prolong the lives of mice with EGFRvIII expressing tumor cells, mainly by inhibiting the growth of the tumor cells in the brains of the mice.

While this invention has been particularly shown and described with references to example embodiments thereof, it will be understood by those skilled in the art that various changes in form and details may be made therein without departing from the scope of the invention encompassed by the appended claims.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.
Altenschmidt, U., Klundt, E. and Groner, B. Adoptive transfer of in vitro-targeted, activated T lymphocytes results in total tumor regression. J Immunol 159 (1997) 5509-5515.
Amy B. Heimberger, Roman Hlatky, Dima Suki, David Yang, Jeff Weinberg, Mark Gilbert, Raymond Sawaya and Kenneth Aldape. Prognostic Effect of Epidermal Growth Factor Receptor and EGFRvIII in Glioblastoma Multiforme Patients. Clinical Cancer Research 2005, 11:1462-1466,
Antony P A, Piccirillo C A, Akpinarli A, et al. CD8+ T-cell immunity against a tumor/self-antigen is augmented by CD4+T helper cells and hindered by naturally occurring T regulatory cells. J Immunol 2005 Mar. 1; 174(5):2591-601.
Baecher-Allan C, Anderson D E. Immune regulation in tumor-bearing hosts. Curr Opin Immunol 2006 April; 18(2):214-9.
Badie B, Schartner J, Paul J, Bartley B, Vorpahl J, and Preston K. Dexamethasone-induced abolition of the inflammatory response in an experimental glioma model: a flow cytometry study. J Neurosurg: 93:634-639.
Beers R, Chowdhury P, Bigner D, Pastan I., Immunotoxins with increased activity against epidermal growth factor receptor vIII-expressing cells produced by antibody phage display. Clin Cancer Res. 2000 July; 6(7):2835-43.
Bigner, D. D., Bigner, S. H., Ponten, J., Westermark, B., Mahaley, M. S., Ruoslahti, E., Herschman, H., Eng, L. F. and Wikstrand, C. J. Heterogeneity of Genotypic and phenotypic characteristics of fifteen permanent cell lines derived from human gliomas. J Neuropathol Exp Neurol 40 (1981) 201-229.
Blehm K N, Spiess P E, Bondaruk J E, Dujka M E, Villares G J, Zhao Y J, Bogler O, Aldape K D, Grossman H B, Adam L, McConkey D J, Czerniak B A, Dinney C P, Bar-Eli M. Mutations within the kinase domain and truncations of the epidermal growth factor receptor are rare events in bladder cancer: implications for therapy. Clin Cancer Res. 2006 Aug. 1; 12(15):4671-7.
Brewer J A, Kanagawa O, Sleckman B P, Muglia U. Thymocyte apoptosis induced by T-cell activation is mediated by glucocorticoids in vivo. J Immunol 2002 Aug. 15; 169(4):1837-43.
Brewer J A, Khor B, Vogt S K, Muglia L M, Fujiwara H, Haegele K E, Sleckman B P, Muglia L J. T-cell glucocorticoid receptor is required to suppress COX-2-mediated lethal immune activation. Nat Med. 2003; 9:1318-22
Brunetti M, Colasante A, Mascetra N, Piantelli M, Musiani P and Aiello F. IL-10 synergized with dexamethasone in inhibiting Human T-cell proliferation. J. Pharm and Experimental Therapeutics. 285:915-919: 1998.
Chakraborty M, Abrams S I, Camphausen K, et al. Irradiation of tumor cells up-regulates fas and enhances CTL lytic activity and CTL adoptive immunotherapy. J Immunol 2003 Jun. 15; 170(12):6338-47.
Chiocca, E. A. Experimental and clinical gene therapies. In Chiocca, E. A. and Breakefield, X. O. (Eds.), Gene Therapy for Neurological Disorders and Brain Tumors. Humana Press Inc., Totowa, N.J., 1998, pp. 191-203.
Coles R M, Mueller S N, Heath W R, Carbone F R, Brooks A G. Progression of armed CTL from draining lymph node to spleen shortly after localized infection with herpes simplex virus 1. J Immunol. 2002 Jan. 15; 168(2):834-8
Deeks, S. G., Wagner, B., Anton, P. A., Mitsuyasu, R. T., Scadden, D. T., Huang, C., Macken, C., Richman, D. D., Christopherson, C., June, C. H., Lazar, R., Broad, D. F., Jalali, S, and Hege, K. M. A phase II randomized study of HIV-specific T-cell gene therapy in subjects with undetectable plasma viremia on combination antiretroviral therapy. Mol Ther 5 (2002) 788-797.
Dobrzanski, M. J., Reome, J. B. and Dutton, R. W. Therapeutic effects of tumor-reactive type 1 and type 2 CD8+ T-cell subpopulations in established pulmonary metastases. J Immunol 162 (1999) 6671-6680.
El Andaloussi A, Lesniak M S. An increase in CD4+CD25+FOXP3+ regulatory T-cells in tumor-infiltrating lymphocytes of human glioblastoma multiforme. Neuro-oncol 2006 May 24.
Fernandes H, Cohen S, Bishayee S: Glycosylation-induced conformational modification positively regulates receptor-receptor association: a study with an aberrant epidermal growth factor receptor (EGFRvIII/DeltaEGFR) expressed in cancer cells. J Biol Chem 276:5375-5383, 2001.
Gattinoni L, Klebanoff C A, Palmer D C, Wrzesinski C, Kerstann K, Yu Z, Finkelstein S E, Theoret M R, Rosenberg S A, Restifo N P. Acquisition of full effector function in vitro paradoxically impairs the in vivo antitumor efficacy of adoptively transferred CD8+ T cells. J Clin Invest. 2005; 115:1616-26.
Garnett C T, Palena C, Chakraborty M, Tsang K Y, Schlom J, Hodge J W. Sublethal irradiation of human tumor cells modulates phenotype resulting in enhanced killing by cytotoxic T lymphocytes. Cancer Res 2004 Nov. 1; 64(21):7985-94.
Gilham D E, O'Neil A, Hughes C, Guest R D, Kirillova N, Lehane M, Hawkins R E. Primary polyclonal human T lymphocytes targeted to carcino-embryonic antigens and neural cell adhesion molecule tumor antigens by CD3zeta-based chimeric immune receptors. J Immunother (1997). 2002 March-April; 25(2):139-51.
Goleva E, Cardona I D, Ou L S, Leung D Y. Factors that regulate naturally occurring T regulatory cell-mediated suppression. J Allergy Clin Immunol 2005 November; 116(5):1094-100.
Graf, M. R., Prins, R. M., Poulsen, G. A. and Merchant, R. E. Contrasting effects of interleukin-2 secretion by rat glioma cells contingent upon anatomical location accelerated tumorigenesis in the central nervous system and complete rejection in the periphery. J Neuroimmunol 140 (2003) 49-60.
Gyobu, H., Tsuji, T., Suzuki, Y., Ohkuri, T., Chamoto, K., Kuroki, M., Miyoshi, H., Kawarada, Y., Katoh, H., Takeshima, T. and Nishimura, T. Generation and targeting of human tumor-specific Tc1 and Th1 cells transduced with a lentivirus containing a chimeric immunoglobulin T-cell receptor. Cancer Res 64 (2004) 1490-1495.
Haynes, N. M., Trapani, J. A., Teng, M. W., Jackson, J. T., Cerruti, L., Jane, S. M., Kershaw, M. H., Smyth, M. J. and Darcy, P. K. Rejection of syngeneic colon carcinoma by CTLs expressing single-chain antibody receptors co-delivering CD28 costimulation. J Immunol 169 (2002) 5780-6.
Heiss J D, Papavassiliou E, Merrill M J, et al. Mechanism of dexamethasone suppression of brain tumor-associated vascular permeability in rats. involvement of the glucocorticoid receptor and vascular permeability factor. J Clin Invest 1996 Sep. 15; 98(6):1400-8.
Jensen M, Tan G, Forman S, Wu A M, Raubitschek A. CD20 is a molecular target for scFvFc:zeta receptor redirected T cells: implications for cellular immunotherapy of CD20+ malignancy. Biol Blood Marrow Transplant. 1998; 4(2):75-83.
Ishikawa E, Tsuboi K, Saijo K, Takano S, Ohno T. X-irradiation to human malignant glioma cells enhances the cytotoxicity of autologous killer lymphocytes under specific conditions. Int J Radiat Oncol Biol Phys 2004 Aug. 1; 59(5):1505-12.
Hombach A, Koch D, Sircar R, Heuser C, Diehl V, Kruis W, Pohl C, Abken H. A chimeric receptor that selectively targets membrane-bound carcinoembryonic antigen (mCEA) in the presence of soluble CEA. Gene Ther. 1999 February; 6(2):300-4.
Horiguchi, H., Hirose, T., Kannuki, S., Nagahiro, S, and Sano, T. Gliosarcoma: an immunohistochemical, ultrastructural and fluorescence in situ hybridization study. Pathol Int 48 (1998) 595-602.
Hwu, P., Yang, J. C., Cowherd, R., Treisman, J., Shafer, G. E., Eshhar, Z. and Rosenberg S A. In vivo antitumor activity of T-cells redirected with chimeric antibody/T-cell receptor genes. Cancer Res 55 (1995) 3369-3373.
Kahlon K S, Brown C, Cooper L J, Raubitschek A, Forman S J, Jensen M C. Specific recognition and killing of glioblastoma multiforme by interleukin 13-zetakine redirected cytolytic T-cells. Cancer Research 64, 9160-9166
Kershaw M, Teng M, Smyth M J and Darcy P K. Supernatural T-cells: Genetic modification of T-cells for Cancer Therapy. Nature Immunology Reviews 5 928 (2005)
Kleihues, P. Subsets of glioblastoma clinical and histological vs. genetic typing. Brain Pathol 8 (1998) 667-668.
Kuan C T, Wikstrand C J, Archer G, Beers R, Pastan I, Zalutsky M R, Bigner D D. Increased binding affinity enhances targeting of glioma xenografts by EGFRvIII-specific scFv. Int J Cancer. 2000 Dec. 15; 88(6):962-9.
Lal A, Glazer C, Martinson H, Friedman H, Archer G, Sampson G, Riggins G. Mutant Epidermal Growth Factor Receptor Up-Regulates Molecular Effectors of Tumor Invasion. Cancer Research 62:3335-3339, 2002
Lamers, C. H., Willemsen, R. A., Luider, B. A., Debets, R., Bolhuis, R. L. Protocol for gene transduction and expansion of human T lymphocytes for clinical immunogene therapy of cancer.” Cancer Gene 9 (2002) 613-623.
Lorimer I A, Keppler-Hafkemeyer A, Beers R A, Pegram C N, Bigner D D, Pastan I: Recombinant immunotoxins specific for a mutant epidermal growth factor receptor: targeting with a single chain antibody variable domain isolated by phage display. Proc Natl Acad Sci USA 93:14815-14820, 1996.
Lutsiak M E, Semnani R T, De Pascalis R, Kashmiri S V, Schlom J, Sabzevari H. Inhibition of CD4(+)25+ T regulatory cell function implicated in enhanced immune response by low-dose cyclophosphamide. Blood 2005 Apr. 1; 105(7):2862-8.
Maher, J., Brentjens, R. J., Gunset G, Riviere I, Sadelain M. Human T-lymphocyte cytotoxicity and proliferation directed by a single chimeric TCRzeta/CD28 receptor. Nat Biotechnol 20 (2002) 70-75.
Maus M V, Thomas A K, Leonard D G, Allman D, Addya K, Schlienger K, Riley J L, June C H. Ex vivo expansion of polyclonal and antigen-specific cytotoxic T lymphocytes by artificial APCs expressing ligands for the T-cell receptor, CD28 and 4-1BB. Nat. Biotechnol. 2002 February; 20(2):143-8.
McGuinness R P, Ge Y, Patel S D, Kashmiri S V, Lee H S, Hand P H, Schlom J, Finer M H, McArthur J G. Anti-tumor activity of human T cells expressing the CC49-zeta chimeric immune receptor. Hum Gene Ther. 1999 Jan. 20; 10(2):165-73.
Mitsuyasu, R. T., Anton, P. A., Deeks, S. G., Scadden, D. T., Connick, E., Downs, M. T., Bakker, A., Roberts, M. R., June, C. H., Jalali, S., Lin, A. A., Pennathur-Das, R. and Hege, K. M. Prolonged survival and tissue trafficking following adoptive transfer of CD4zeta gene-modified autologous CD4(+) and CD8(+) T-cells in human immunodeficiency virus-infected subjects. Blood 96 (2000) 785-793.
Modjtahedi H, Moscatello D K, Box G, Green M, Shotton C, Lamb D J, Reynolds L J, Wong A J, Dean C, Thomas H, Eccles S. Targeting of cells expressing wild-type EGFR and type-III mutant EGFR (EGFRvIII) by anti-EGFR MAb ICR62: a two-pronged attack for tumour therapy. Int J Cancer. 2003 Jun. 10; 105(2):273-80.
Moscatello D K, Holgado-Madruga M, Godwin A K, Ramirez G, Gunn G, Zoltick P W, Biegel J A, Hayes R L, Wong A J: Frequent expression of a mutant epidermal growth factor receptor in multiple human tumors. Cancer Res 55:5536-5539, 1995.
Motoyoshi Y, Kaminoda K, Saitoh O, et al. Different mechanisms for anti-tumor effects of low- and high-dose cyclophosphamide. Oncol Rep 2006 July; 16(1):141-6.
Ng, H.-K. and Lam, P. The molecular genetics of CNS tumors. Pathology 30 (1998) 196-202.
Niederman, T. M., Ghogawala, Z., Carter, B. S., Tompkins, H. S., Russell, M. M. and Mulligan, R. C. Antitumor activity of cytotoxic T lymphocytes engineered to target vascular endothelial growth factor receptors. Proc Natl Acad Sci USA 99 (2002) 7009-7014.
Nishikawa R, Sugiyama T, Narita Y, Furnari F, Cavenee W K, Matsutani M. Immunohistochemical analysis of the mutant epidermal growth factor, deltaEGFR, in glioblastoma. Brain Tumor Pathol. 2004; 21(2):53-6.
Pasternack M S, Eisen H N. A novel serine esterase expressed by cytotoxic T lymphocytes. Nature 1985 Apr. 25-May 1; 314(6013):743-5.
Pasternack M S, Verret C R, Liu M A, Eisen H N. Serine esterase in cytolytic T lymphocytes. Nature 1986 Aug. 21-27; 322(6081):740-3.
Pinthus J H, Waks T, Malina V, Kaufman-Francis K, Harmelin A, Aizenberg I, Kanety H, Ramon J, Eshhar Z. Adoptive immunotherapy of prostate cancer bone lesions using redirected effector lymphocytes. J Clin Invest. 2004 December; 114(12):1774-81. Plautz G E, Inoue M, Shu S. Defining the synergistic effects of irradiation and T-cell immunotherapy for murine intracranial tumors. Cell Immunol 1996 Aug. 1; 171(2):277-84.
Ramnarain D B, Park S, Lee D Y, Hatanpaa K J, Scoggin S O, Otu H, Libermann T A, Raisanen J M, Ashfaq R, Wong E T, Wu J, Elliott R, Habib A A. Differential gene expression analysis reveals generation of an autocrine loop by a mutant epidermal growth factor receptor in glioma cells. Cancer Res. 2006 Jan. 15; 66(2):867-74.
Sampson J H, Akabani G, Archer G E, Bigner D D, Berger M S, Friedman A H, Friedman H S, Herndon J E, 2nd, Kunwar S, Marcus S, McLendon R E, Paolino A, Penne K, Provenzale J, Quinn J, Reardon D A, Rich J, Stenzel T, Tourt-Uhlig S, Wikstrand C, Wong T, Williams R, Yuan F, Zalutsky M R, Pastan I: Progress report of a Phase I study of the intracerebral microinfusion of a recombinant chimeric protein composed of transforming growth factor (TGF)-alpha and a mutated form of the Pseudomonas exotoxin termed PE-38 (TP-38) for the treatment of malignant brain tumors. J Neurooncol 65:27-35, 2003.
Sampson J H, Crotty L E, Lee S, Archer G E, Ashley D M, Wikstrand C J, Hale L P, Small C, Dranoff G, Friedman A H, Friedman H S, Bigner D D: Unarmed, tumor-specific monoclonal antibody effectively treats brain tumors. Proc Natl Acad Sci USA 97:7503-7508, 2000.
Schmidt M, Vakalopoulou E, Schneider D W, Wels W. Construction and functional characterization of scFv (14E1)-ETA—a novel, highly potent antibody-toxin specific for the EGF receptor. Br J Cancer. 1997; 75(11):1575-84.
Shinojima N, Tada K, Shiraishi S, Kamiryo T, Kochi M, Nakamura H, Makino K, Saya H, Hirano H, Kuratsu J, Oka K, Ishimaru Y, Ushio Y: Prognostic value of epidermal growth factor receptor in patients with glioblastoma multiforme. Cancer Res 63:6962-6970, 2003
Steinbach J P, Wolburg H, Klumpp A, Weller M. Hypoxia sensitizes human malignant glioma cells towards CD95L-induced cell death. J Neurochem 2005 March; 92(6):1340-9.
Sok J C, Coppelli F M, Thomas S M, Lango M N, Xi S, Hunt J L, Freilino M L, Graner M W, Wikstrand C J, Bigner D D, Gooding W E, Furnari F B, Grandis J R. Mutant epidermal growth factor receptor (EGFRvIII) contributes to head and neck cancer growth and resistance to EGFR targeting. Clin Cancer Res. 2006 Sep. 1; 12(17):5064-73.
Tang C K, Gong X Q, Moscatello D K, Wong A J, Lippman M E: Epidermal growth factor receptor vIII enhances tumorigenicity in human breast cancer. Cancer Res 60:3081-3087, 2000.
Tran A C, Zhang D, Byrn R, Roberts M R. Chimeric zeta-receptors direct human natural killer (NK) effector function to permit killing of NK-resistant tumor cells and HIV-infected T lymphocytes. J Immunol. 1995 Jul. 15; 155(2):1000-9.
Turatti F, Figini M, Balladore E, Alberti P, Casalini P, Marks J D, Canevari S, Mezzanzanica D. Redirected Activity of Human Antitumor Chimeric Immune Receptors is Governed by Antigen and Receptor Expression Levels and Affinity of Interaction. J Immunother. 2007 30: 684-693.
von Zons, P., Crowley-Nowick, P., Friberg D, Bell M, Koldovsky U, Whiteside T L. Comparison of europium and chromium release assays cytotoxicity in healthy individuals and patients with cervical carcinoma. Clin Diagn Lab Immunol 4 (1997) 202-207.
Waldmann T A. Effective cancer therapy through immunomodulation. Annu Rev Med 2006; 57:65-81.
Wang, J., Press, O. W., Lindgren, C. G., Greenberg, P., Riddell, S., Qian, X., Laugen, C., Raubitschek, A., Forman, S. J. and Jensen, M. C. Cellular immunotherapy for follicular lymphoma using genetically modified CD20-specific CD8+ cytotoxic T lymphocytes. Mol Ther 9 (2004) 577-586.
Xia S, Rosen E M, Laterra J. Sensitization of glioma cells to fas-dependent apoptosis by chemotherapy-induced oxidative stress. Cancer Res 2005 Jun. 15; 65(12):5248-55.
Yan Y, Johnson B, Orentas R. Murine Cd8 lymphocytes expansion in vitro by artificial antigen-presenting cells expressing CD1378L (4-1BBL) is superior to CD28, and CD137L expressed on neuroblastoma cells expands CD8 tumor reactive effector cells in vivo Immunology. 2004 May; 112(1):105-16.
Zhang Q, Yang X, Pins M, Javonovic B, Juzel T, Kim S J, Parijs L K, Greenberg N M, Liu V, Guo Y, Lee C. Adoptive transfer of tumor reactive TGF Beta Insensitive CD8+. T-cells: Eradication of Autologous Mouse Prostate Cancer. Cancer Res 2005; 65: 1761-9.

1. A nucleic acid encoding a chimeric EGFRvIII T cell receptor, said chimeric EGFRvIII T cell receptor comprising (a) an extracellular domain comprising an EGFRvIII binding portion, (b) a middle portion comprising a transmembrane domain (TM) derived from a human protein, and (c) an intracellular domain comprising a human T-cell receptor zeta chain.

2. The nucleic acid of claim 1, wherein said EGFRvIII binding portion of the EGFRvIII T cell receptor is an MR1 single chain antibody antigen-binding domain or an MR1-1 single chain antibody antigen-binding domain.

3. The nucleic acid of claim 2, wherein said middle portion of the chimeric EGFRvIII T cell receptor is selected from a group consisting of:
a. a human CD8α hinge and TM;
b. a human CD28 protein fragment comprising an extracellular domain, a TM, and an intracellular domain;
c. a fusion protein comprising a human CD8α hinge and TM, a human CD28 intracellular domain and a human OX40 intracellular domain; and
d. a fusion protein comprising a human CD8α hinge, a TM and an intracellular signaling domain of a human CD28, and an intracellular signaling domain of a human OX40.

4. The nucleic acid of claim 3, wherein the nucleic acid is SEQ. ID. No. 1, 3, 5, 9, 11, or 13.

5. (canceled)

6. (canceled)

7. A cell comprising the nucleic acid of claim 3.

8. The cell of claim 7, wherein the cell is a T-cell.

9. A chimeric EGFRvIII targeting T cell receptor encoded by a nucleic acid of claim 3.

10. The chimeric EGFRvIII targeting T cell receptor of claim 9, wherein the nucleic acid is SEQ. ID. No. 2, 4, 6, 10, 12, or 14.

11. (canceled)

12. (canceled)

13. (canceled)

14. (canceled)

15. (canceled)
